(19) United States
(12) Patent Application Publication
(10) Pub. No.: US 2026/0370934 A1
(43) Pub. Date: Nov. 19, 2026

(54) INFORMATION PROCESSING SYSTEM, INFORMATION PROCESSING METHOD, AND NON-TRANSITORY RECORDING MEDIUM

(71) Applicant: NEC Corporation, Tokyo (JP)

(72) Inventors: Chisato FUNAYAMA, Tokyo (JP); Takahiro TOIZUMI, Tokyo (JP); Yuka OGINO, Tokyo (JP); Yuho SHOJI, Tokyo (JP); Hirokazu AKIBA, Tokyo (JP); Shinji OOMINATO, Tokyo (JP); Hironori MOROOKA, Tokyo (JP); Megumi HASHIMOTO, Tokyo (JP); Yoshihiro KAJIKI, Tokyo (JP); Toshinobu OGATSU, Tokyo (JP)

(73) Assignee: NEC Corporation, Tokyo (JP)

(21) Appl. No.: 19/132,415

(22) PCT Filed: Nov. 29, 2022
(86) PCT No.: PCT/JP2022/044025

§ 371 Date: May 23, 2025

(51) Int. Cl. H04N 23/60; G06V 10/98; G06V 40/18; G06V 40/50

(52) U.S. Cl. CPC H04N 23/64; G06V 10/993; G06V 40/197; G06V 40/50

(57) ABSTRACT

An information processing system includes: an image capturing unit that captures an eye image including an iris of an image capturing target; and an output unit that outputs first guide information for capturing the eye image that allows iris recognition, to a photographer who is different from the image capturing target, in response to the eye image being captured. According to the information processing system, the guide information is outputted to the photographer. Therefore, even the photographer who is not accustomed to capture images of the iris is able to capture the eye image suitable for the iris recognition.

### TECHNICAL FIELD

The present disclosure relates to technical fields of an information processing system, an information processing method, and a recording medium.

### BACKGROUND ART

A known system of this type captures an image including an iris of a target (hereinafter referred to as an “iris image”) to use, for example, in biometric authentication or the like. Patent Literature 1 discloses a technique/technology in which doctors, nurses, or the like capture the iris image of the target in order to output rescue information. Patent Literature 2 discloses a technique/technology in which a guide frame for alignment is displayed to have a user himself capture the iris image. Patent Literature 3 discloses a technique/technology of determining whether or not an image used for iris recognition or the like is of sufficient quality.

### CITATION LIST

### Patent Literature

Patent Literature 1: International Publication No. WO2020/079719
Patent Literature 2: JP2017-54179A
Patent Literature 3: JP2016-51482A

### SUMMARY

### Technical Problem

The present disclosure aims to improve the techniques/technologies disclosed in Citation List.

### Solution to Problem

An information processing system according to an example aspect of the present disclosure includes: an image capturing unit that captures an eye image including an iris of an image capturing target; and an output unit that outputs first guide information for capturing the eye image that allows iris recognition, to a photographer who is different from the image capturing target, in response to the eye image being captured.

An information processing method according to an example aspect of the present disclosure is an information processing method that is executed by at least one computer, the information processing method including: capturing an eye image including an iris of an image capturing target; and outputting first guide information for capturing the eye image that allows iris recognition, to a photographer who is different from the image capturing target, in response to the eye image being captured.

A recording medium according to an example aspect of the present disclosure is a recording medium on which a computer program that allows at least one computer to execute an information processing method is recorded, the information processing method including: capturing an eye image including an iris of an image capturing target; and outputting first guide information for capturing the eye image that allows iris recognition, to a photographer who is different from the image capturing target, in response to the eye image being captured.

### BRIEF DESCRIPTION OF DRAWINGS

FIG. 1 is a block diagram illustrating a hardware configuration of an information processing system according to a first example embodiment.

FIG. 2 is a block diagram illustrating a functional configuration of the information processing system according to the first example embodiment.

FIG. 3 is a flowchart illustrating a flow of operation of the information processing system according to the first example embodiment.

FIG. 4 is a block diagram illustrating a functional configuration of an information processing system according to a second example embodiment.

FIG. 5 is a side view illustrating an arrangement example of an image capturing unit and a display unit according to the information processing system according to the second example embodiment.

FIG. 6 is a plan view illustrating a display example of an iris capturing frame in the information processing system according to a third example embodiment.

FIG. 7 is a plan view illustrating a display example of the iris capturing frame and a pupil frame in the information processing system according to the third example embodiment.

FIG. 8 is a plan view illustrating an operation example at the time of displaying the iris capturing frame in an information processing system according to a fourth example embodiment.

FIG. 9 is a plan view illustrating a display example of first guide information in an information processing system according to a fifth example embodiment.

FIG. 10 is a block diagram illustrating a functional configuration of an information processing system according to a sixth example embodiment.

FIG. 11 is a flowchart illustrating a flow of operation of the information processing system according to the sixth example embodiment.

FIG. 12 is a flowchart illustrating a flow of operation of an information processing system according to a seventh example embodiment.

FIG. 13 is a plan view illustrating an output example of the first guide information in an information processing system according to an eighth example embodiment.

FIG. 14 is a plan view illustrating a modification of the first guide information in the information processing system according to the eighth example embodiment.

FIG. 15 is a block diagram illustrating a functional configuration of an information processing system according to a ninth example embodiment.

FIG. 16 is a flowchart illustrating a flow of operation of the information processing system according to the ninth example embodiment.

FIG. 17 is a conceptual diagram illustrating an output example of the first guide information and second guide information by an information processing system according to a 10th example embodiment.

FIG. 18 is a block diagram illustrating a functional configuration of an information processing system according to an 11th example embodiment.

FIG. 19 is a flowchart illustrating a flow of an adjustment determining operation of the information processing system according to the 11th example embodiment.

FIG. 20 is a conceptual diagram illustrating an output example of the first guide information and the second guide information by the information processing system according to the 11th example embodiment.

FIG. 21 is a flowchart illustrating a flow of an adjustment determining operation of an information processing system according to a 12th example embodiment.

FIG. 22 is a conceptual diagram illustrating an output example of the first guide information and the second guide information by the information processing system according to the 12th example embodiment.

FIG. 23 is a block diagram illustrating a functional configuration of an information processing system according to a 13th example embodiment.

FIG. 24 is a conceptual diagram illustrating a cooperator identifying operation by the information processing system according to the 13th example embodiment.

FIG. 25 is a flowchart illustrating a flow of operation of the information processing system according to the 13th example embodiment.

FIG. 26 is a block diagram illustrating a functional configuration of an information processing system according to a 14th example embodiment.

FIG. 27 is a flowchart illustrating a flow of a photographer determining operation by the information processing system according to the 14th example embodiment.

FIG. 28 is a plan view illustrating a display example of the first guide information by an information processing system according to a 15th example embodiment.

FIG. 29 is a block diagram illustrating a functional configuration of an information processing system according to a 16th example embodiment.

FIG. 30 is a flowchart illustrating a flow of a skill level determining operation by the information processing system according to the 16th example embodiment.

FIG. 31 is a block diagram illustrating a functional configuration of an information processing system according to a 17th example embodiment.

FIG. 32 is a flowchart illustrating a flow of an image registering operation by the information processing system according to the 17th example embodiment.

### DESCRIPTION OF EXAMPLE EMBODIMENTS

Hereinafter, an information processing system, an information processing method, and a recording medium according to example embodiments will be described with reference to the drawings.

### First Example Embodiment

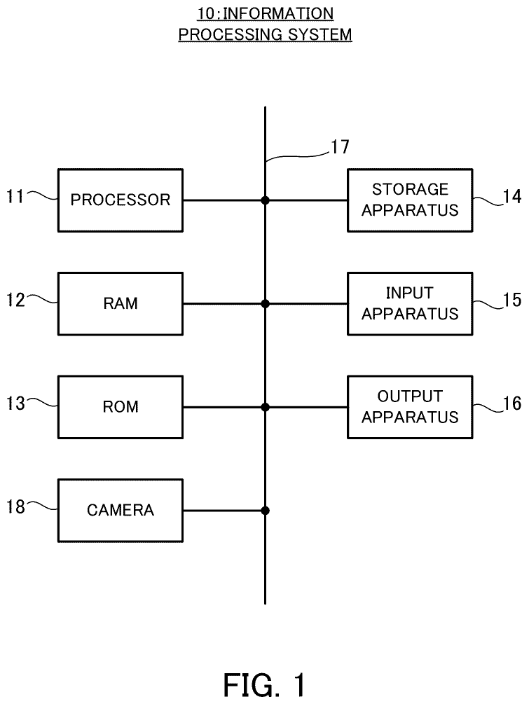

An information processing system according to a first example embodiment will be described with reference to FIG. 1 to FIG. 3.

### (Hardware Configuration)

First, with reference to FIG. 1, a hardware configuration of the information processing system according to the first example embodiment will be described. FIG. 1 is a block diagram illustrating the hardware configuration of the information processing system according to the first example embodiment.

As illustrated in FIG. 1, an information processing system 10 according to the first embodiment includes a processor 11, a processor 11, a RAM (Random Access Memory) 12, a ROM (Read Only Memory) 13, and a storage apparatus 14. The information processing system 10 may also include an input apparatus 15, an output apparatus 16, and a camera 18. The processor 11, the RAM 12, the ROM 13, the storage apparatus 14, the input apparatus 15, the output apparatus 16, and the camera 18 are connected via a data bus 17.

The processor 11 reads a computer program. For example, the processor 11 is configured to read a computer program stored in at least one of the RAM 12, the ROM 13, and the storage apparatus 14. Alternatively, the processor 11 may read a computer program stored on a computer-readable recording medium, by using a not-illustrated recording medium reading apparatus. The processor 11 may also acquire (i.e., may read) a computer program from a not-illustrated apparatus disposed outside the information processing system 10 via a network interface. The processor 11 controls the RAM 12, the storage apparatus 14, the input apparatus 15, and the output apparatus 16 by executing the read computer program. Especially in the present example embodiment, when the processor 11 executes the read computer program, a functional block for outputting guide information when an iris image is captured, is realized in the processor 11. That is, the processor 11 may function as a controller for executing each control in the information processing system 10.

The processor 11 may be configured as, for example, a CPU (central processing unit), a GPU (graphics processing unit), a FPGA (field-programmable gate array), a DSP (Digital Signal Processor), an ASIC (Application Specific Integrated Circuit), and a quantum processor. The processor 11 may be one of them, or may use a plurality of them in parallel.

The RAM 12 temporarily stores the computer program to be executed by the processor 11. The RAM 12 temporarily stores data that are temporarily used by the processor 11 when the processor 11 executes the computer program. The RAM 12 may be, for example, a D-RAM (Dynamic Random Access Memory) or a SRAM (Static Random Access Memory). Furthermore, another type of volatile memory may be used in place of the RAM 12.

The ROM 13 stores the computer program to be executed by the processor 11. The ROM 13 may also store fixed data. The ROM 13 may be, for example, a P-ROM (Programmable Read Only Memory) or an EPROM (Erasable Read Only Memory). In addition, another type of nonvolatile memory may be used in place of the ROM 13.

The storage apparatus 14 stores data that are stored by the information processing system 10 for a long time. The storage apparatus 14 may operate as a temporary/transitory storage apparatus of the processor 11. The storage apparatus 14 may include, for example, at least one of a hard disk apparatus, a magneto-optical disk apparatus, a SSD (Solid State Drive), and a disk array apparatus.

The input apparatus 15 is an apparatus that receives an input instruction from a user of the information processing system 10. The input apparatus 15 may include, for example, at least one of a keyboard, a mouse, and a touch panel. The input apparatus 15 may be configured as a portable terminal such as a smartphone and a tablet. The input apparatus 15 may be an apparatus that allows audio input/voice input, including a microphone, for example.

The output apparatus 16 is an apparatus that outputs information about the information processing system 10 to the outside. For example, the output apparatus 16 may be a display device (e.g., a display) that is configured to display the information about the information processing system 10. The output apparatus 16 may also be a speaker or the like that is configured to audio-output the information about the information processing system 10. The output apparatus 16 may be configured as a portable terminal such as a smartphone and a tablet. The output apparatus 16 may also be an apparatus that outputs information in a format other than an image. For example, the output apparatus 16 may be a speaker that audio-outputs the information about the information processing system 10.

The camera 18 is configured to capture an image or a video/moving image. The type of the camera 18 is not particularly limited, and may be a visible light camera or a near-infrared camera, for example. The camera 18 may be mounted on a terminal (e.g., a smartphone) carried/owned by the user, for example. In addition, the information processing system 10 may include a plurality of cameras 18. In this case, the plurality of cameras may be different types of cameras (e.g., cameras with different image capturing ranges, image capturing angles, image capturing qualities, etc.).

Although FIG. 1 illustrates an example of the information processing system 10 including a plurality of apparatuses, all or a part of the functions of these apparatuses may be realized by a single apparatus (an information processing apparatus). In that case, the information processing apparatus may include, for example, only the processor 11, the RAM 12, and the ROM 13. The other components (i.e., the storage apparatus 14, the input apparatus 15, the output apparatus 16, and the camera 18) may be provided in an external apparatus connected to the information processing apparatus. In addition, in the information processing apparatus, a part of an arithmetic function may be realized by an external apparatus (e.g., an external server or cloud, etc.).

### (Functional Configuration)

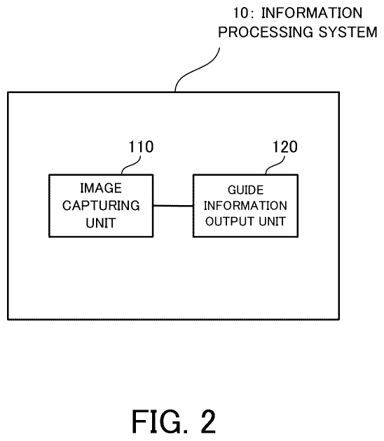

Next, with reference to FIG. 2, a description will be given of a functional configuration of the information processing system 10 according to the first example embodiment. FIG. 2 is a block diagram illustrating the functional configuration of the information processing system according to the first example embodiment.

As illustrated in FIG. 2, the information processing system 10 according to the first example embodiment includes, as components for realizing the functions thereof, an image capturing unit 110 and a guide information output unit 120. The image capturing unit 110 may include, for example, the camera 18 described above. The guide information output unit 120 may be a processing block realized by the processor 11 (see FIG. 1), for example.

The image capturing unit 110 is configured to capture an eye image including an iris of a target. The term “target” here is not limited to humans, and may include animals such as, for example, dogs and cats, and robots or the like. The image captured by the image capturing unit 110 may be, for example, an image to be used for iris recognition. Specifically, the image capturing unit 110 may captures a registration image in the iris recognition (i.e., an image of a person himself registered in advance) and an image captured in the iris recognition (i.e., an image for authenticating an identity of the person in question by collating/verifying it with the registration image).

The guide information output unit 120 is configured to output first guide information when the eye image is captured by the image capturing unit 110. In addition, it is assumed that in the information processing system 10 in the present example embodiment, an iris image is captured by a photographer/cameraman who is different from an image capturing target, and the guide information output unit 120 outputs the first guide information to the photographer who is different from the image capturing target. The guide information output unit 120 outputs the first guide information, as information for capturing an image that allows the iris recognition. The term “image that allows the iris recognition” here refers to an image of sufficient quality to enable normal iris recognition, and for example, a quality criterion that satisfies a predetermined recognition performance may be set through prior simulation or the like. Examples of the image of sufficient quality may be an image with high resolution, an image with a large iris area (e.g., an image of the eyes wide open, an image of the iris captured close to a camera, etc.), an image including an entire iris, and the like.

The first guide information may include information for improving the quality of the eye image, for example. Specifically, the first guide information may include information prompting the user to change the position of the camera 18 or adjust camera parameters, or the like. The first guide information may include a plurality of pieces of information. In that case, the plurality of pieces of information may be outputted, simultaneously or sequentially. The first guide information may be information outputted via the output apparatus 16 described above, for example. More specifically, the first guide information may be displayed on a display, or may be audio-outputted from a speaker. Alternatively, the first guide information may be information outputted by using a vibration function. A more specific example of the first guide information will be described in detail in another example embodiment described later.

### (Flow of Operation)

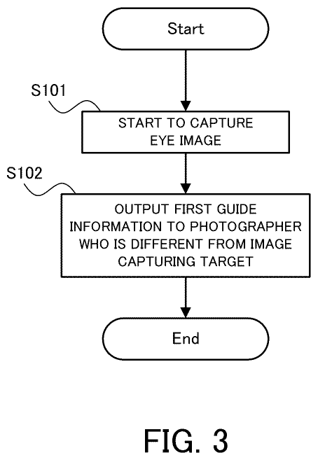

Next, with reference to FIG. 3, a description will be given of a flow of operation of the information processing system 10 according to the first example embodiment. FIG. 3 is a flowchart illustrating the flow of the operation of the information processing system according to the first example embodiment.

As illustrated in FIG. 3, when the operation of the information processing system 10 according to the first example embodiment is started, the image capturing unit 110 first starts to capture the eye image (step S101). Note that the start of the image capturing here may not refer to a time of each eye image being captured by the photographer's operation (e.g. pressing a shutter button, etc.), but may be a time in which the photographer holds the camera 18 toward the image capturing target and in which an area in the image capturing range of the camera 18 starts to be displayed as a live view image.

Next, the guide information output unit 120 outputs the first guide information to the photographer (step S102). The first guide information may be generated and outputted, based on the image being captured by the image capturing unit 110. That is, the first guide information may be information that varies depending on a current image capturing environment or the like. Given here is an example of outputting the first guide information after starting to capture the eye image, but the first guide information may be outputted before starting to capture the eye image.

### (Technical Effect)

Next, a description will be given of a technical effect obtained by the information processing system 10 according to the first example embodiment.

As described in FIG. 1 to FIG. 3, in the information processing system 10 according to the first example embodiment, the first guide information is outputted to the photographer when the eye image is captured. In this way, even the photographer who is not accustomed to capture images of the iris is able to capture the eye image that allows the iris recognition with reference to the first guide information.

### Second Example Embodiment

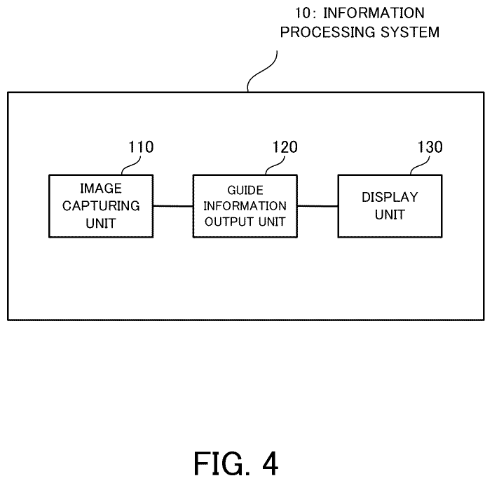

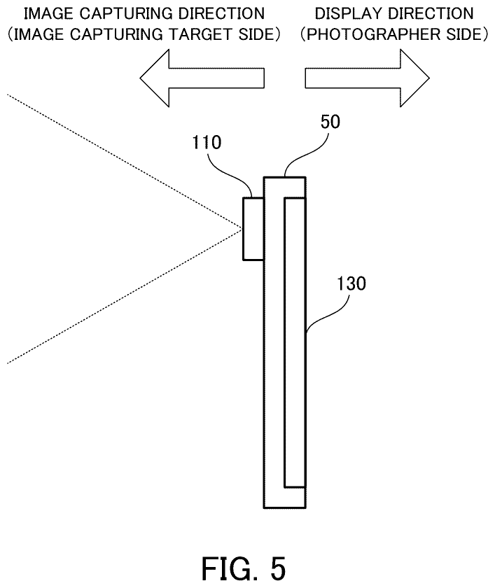

The information processing system 10 according to a second example embodiment will be described with reference to FIG. 4 and FIG. 5. The second example embodiment partially differs from the first example embodiment only in its configuration, and may be the same as the first example embodiment in the other parts. For this reason, a part differing from the already described first example embodiment will be described in detail below, and a description of the other overlapping parts will be omitted as appropriate.

### (Functional Configuration)

First, with reference to FIG. 4, a description will be given of a functional configuration of the information processing system 10 according to the second example embodiment. FIG. 4 is a block diagram illustrating the functional configuration of the information processing system according to the second example embodiment. In FIG. 4, the same components as those illustrated in FIG. 2 carry the same reference numerals.

As illustrated in FIG. 4, the information processing system 10 according to the second example embodiment includes, as components for realizing the functions thereof, the image capturing unit 110, the guide information output unit 120, and a display unit 130. That is, the information processing system 10 according to the second example embodiment includes the display unit 130, in addition to the configuration in the first example embodiment (see FIG. 2). The display unit 130 may be configured as the output apparatus 16 (see FIG. 1), for example. The display unit 130 according to the present example embodiment is particularly configured to display the first guide information. For example, the display unit 130 may be configured to display messages, icons, marks, or the like, indicated by the first guide information.

### (Arrangement Configuration)

Next, with reference to FIG. 5, a description will be given of the arrangement of the image capturing unit 110 and the display unit 130 described above. FIG. 5 is a side view illustrating an arrangement example of the image capturing unit and the display unit in the information processing system according to the second example embodiment.

As illustrated in FIG. 5, the image capturing unit 110 and display unit 130 according to the second example embodiment may be mounted on a same smartphone 50. For example, the image capturing unit 110 may be a main camera mounted on the smartphone 50. The display unit 130 may be a main display mounted on the smartphone 50.

In the information processing system according to the second example embodiment, in particular, the image capturing unit 110 is disposed on a rear side of the display 130. That is, the arrangement configuration is such that an image capturing direction of the image capturing unit 110 and a display direction of the display 130 are opposite to each other. Although an example given here indicates that the image capturing direction of the image capturing unit 110 and the display direction of the display unit 130 are opposite to each other by 180 degrees, these directions may not necessarily be 180 degrees opposite. That is, the “rear side” in the present example embodiment may not mean that the directions are exactly 180 degrees opposite, but rather that a certain amount of margin is allowed, and even in such a case, a technical effect described below may be obtained to a reasonable extent.

### (Technical Effect)

Next, a description will be given of a technical effect obtained by the information processing system 10 according to the second example embodiment.

As described in FIG. 4 and FIG. 5, in the information processing system 10 according to the second example embodiment, the image capturing unit 110 that captures the image capturing target is disposed on the rear side of the display unit 130 that displays the first guide information. In this way, it is possible to properly output the first guide information to the photographer who holds the image capturing unit 110 toward the image capturing target. In addition, in the terminal such as the smartphone 50 described above, not only a rear camera (a so-called “out camera” in Japanese) such as the image capturing unit 110, but also a front camera (so-called an “in camera” in Japanese) may be provided on the display 130 side; however, the image quality of this front camera is often lower than that of the rear camera. In the present example embodiment, however, even in the case of using the above-mentioned terminal, it is possible to output the first guide information to the photographer, while the rear camera, which can capture high-quality images, is pointed at the image capturing target.

### Third Example Embodiment

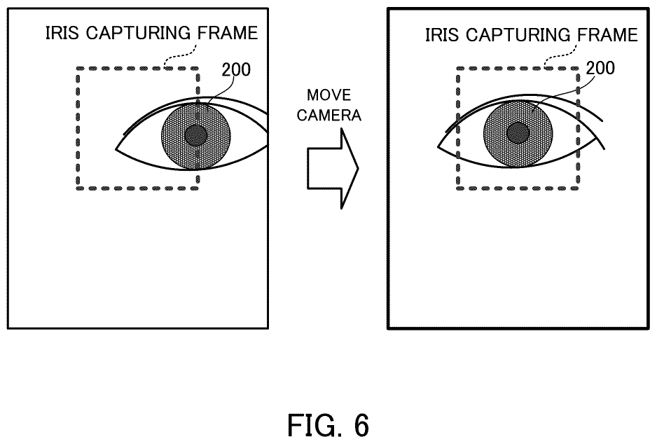

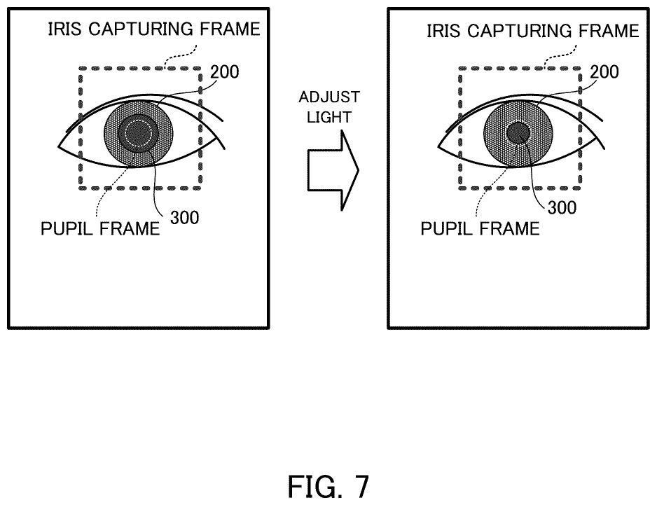

The information processing system 10 according to a third example embodiment will be described with reference to FIG. 6 and FIG. 7. The third example embodiment describes a specific example of the first guide information, and may be the same as the first and second example embodiments in the system configuration and overall flow of operation, or the like. For this reason, a part differing from each of the example embodiments described above will be described in detail below, and a description of the other overlapping parts will be omitted as appropriate.

### (Iris Capturing Frame)

First, with reference to FIG. 6, a description will be given of an iris capturing frame displayed by the information processing system 10 according to the third example embodiment. FIG. 6 is a plan view illustrating a display example of the iris capturing frame in the information processing system according to the third example embodiment.

As illustrated in FIG. 6, in the information processing system 10 according to the third example embodiment, the iris capturing frame may be displayed on the display unit 130 by the first guide information. The iris capturing frame is displayed as a guide for capturing the eye image that allows the iris recognition. The iris capturing frame is set such that the eye image that allows the iris recognition may be captured by capturing the iris 200 within the frame. More specifically, a position of the iris capturing frame being displayed may correspond to an appropriate position of the iris 200 in the image. In addition, a size of the iris capturing frame may correspond to an appropriate size of the iris 200 in the image. The first guide information may include information about the position and size of the iris capturing frame, in order to display the iris capturing frame.

The iris capturing frame is superimposed and displayed on a captured image. The captured image here may be a live view image displayed in real time. By superimposing and displaying the iris capturing frame on the captured image in this way, the photographer is easily able to understand how to move the image capturing unit 110 (i.e., the camera 18) to include the iris 200 within the iris capturing frame. For example, as illustrated in FIG. 6, in a case where the iris 200 of the image capturing target is captured with it shifted to the right from the iris capturing frame, the photographer may move the camera 18 to the right to capture the iris 200 of the image capturing target within the iris capturing frame.

### (Pupil Frame)

Next, with reference to FIG. 7, a description will be given of a pupil frame displayed by the information processing system 10 according to the third example embodiment. FIG. 7 is a plan view illustrating a display example of the iris capturing frame and the pupil frame in the information processing system according to the third example embodiment.

As illustrated in FIG. 7, in the information processing system 10 according to the third example embodiment, the pupil frame may be displayed in addition to the iris capturing frame described above. In this case, the first guide information may include information for displaying the pupil frame, in addition to the information for displaying the iris capturing frame.

The pupil frame is set such that the eye image that allows the iris recognition may be captured by including a pupil 300 within the frame. More specifically, a position of the pupil frame being displayed may be intended to achieve an appropriate position of the iris 200 in the image. For example, the position of the pupil frame may be set such that the pupil 300 is included within the frame, by which the iris 200 located therearound is in an appropriate position. In addition, a size of the pupil frame may be intended to achieve an appropriate state of the iris 200 in the image. For example, the size of the pupil frame may be set such that the pupil 300 is included within the frame, by which the iris 200 located therearound becomes clearly visible (e.g., in a state in which the contraction of the iris 200 due to the dilation of the pupil 300 is reduced/controlled).

In a case where the pupil 300 is dilated to a size that does not fit within the pupil frame, for example, lighting intensity may be increased to perform control of constricting the pupil 300. For example, when the image is captured by the smartphone 50 illustrated in FIG. 5, the information processing system 10 may cause the pupil to be constricted by increasing light intensity of a light source mounted on the smartphone 50.

### (Technical Effect)

Next, a description will be given of a technical effect obtained by the information processing system 10 according to the third example embodiment.

As described in FIG. 6 and FIG. 7, in the information processing system 10 according to the third example embodiment, the iris capturing frame is displayed by the first guide information. By displaying the iris capturing frame, it is possible to capture the eye image including an appropriate size of iris in an appropriate position. In addition, by displaying the pupil frame in addition to the iris capturing frame, it is possible to adjust the position and size of the iris with higher accuracy.

### Fourth Example Embodiment

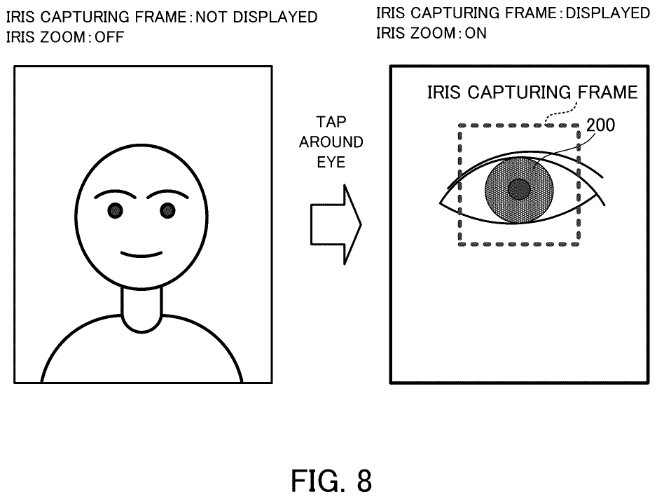

The information processing system 10 according to a fourth example embodiment will be described with reference to FIG. 8. The fourth example embodiment describes an operation example at the time of displaying the iris capturing frame described in the third example embodiment, and may be the same as the third example embodiment in the other parts. For this reason, a part differing from each of the example embodiments described above will be described in detail below, and a description of the other overlapping parts will be omitted as appropriate.

### (Frame Display Operation)

First, with reference to FIG. 8, a description will be given of a frame display operation (i.e., an operation at the time of displaying the iris capturing frame) in the information processing system 10 according to the fourth example embodiment. FIG. 8 is a plan view illustrating an operation example at the time of displaying the iris capturing frame in the information processing system according to the fourth example embodiment.

As illustrated in FIG. 8, let us assume that an image including an entire face of the image capturing target is captured by the image capturing unit 110. At this stage, the iris capturing frame is not displayed. Here, when the photographer taps a part near the eyes of the target (i.e., a part of the display unit 130 where the eyes of the target are displayed), the iris capturing frame is displayed, and the image capturing unit 110 zooms in the tapped part (i.e., around the eyes of the image capturing target). This makes it easier to include the iris 200 of the image capturing target within the iris capturing frame.

### (Technical Effect)

Next, a description will be given of a technical effect obtained by the information processing system 10 according to the fourth example embodiment.

As described in FIG. 8, in the information processing system 10 according to the fourth example embodiment, the iris capturing frame is displayed and the part around the eyes is zoomed in, in response to the operation for the part around the eye of the image capturing target. In this way, it is possible to switch into an appropriate state in which the iris is capturable, with a simple operation.

### Fifth Example Embodiment

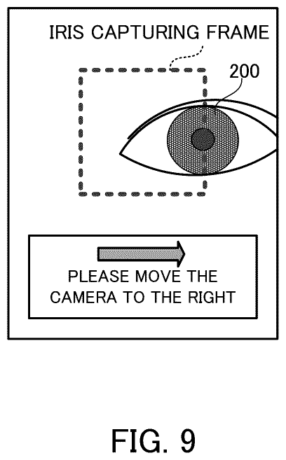

The information processing system 10 according to a fifth example embodiment will be described with reference to FIG. 9. The fifth example embodiment describes a specific example of the first guide information, and may be the same as the first to fourth example embodiments in the other parts. For this reason, a part differing from each of the example embodiments described above will be described in detail below, and a description of the other overlapping parts will be omitted as appropriate.

### (Display of Moving Direction)

First, with reference to FIG. 9, a specific description will be given of the first guide information displayed by the information processing system 10 according to the fifth example embodiment. FIG. 9 is a plan view illustrating a display example of the first guide information in the information processing system according to the fifth example embodiment.

As illustrated in FIG. 9, in the information processing system 10 according to the fifth example embodiment, the first guide information is outputted as information indicating a moving direction of the image capturing unit 110 (i.e., the camera 18). More specifically, the first guide information includes information indicating in which direction the image capturing range of the image capturing unit 110 should be moved in order to capture the iris 200 of the image capturing target within the iris capturing frame.

For example, as illustrated in FIG. 9, in a case where the iris 200 of the image capturing target is captured with it shifted to the right from the iris capturing frame, a message of “Please move the camera to the right” and an arrow pointing to the right may be displayed based on the first guide information. The first guide information may include information for audio-outputting the message as described above. Furthermore, in a case of using a terminal with a vibration function, such as the smartphone 50, the first guide information may indicate a direction of moving the camera 18 by vibration. For example, in a case of moving the camera 18 to the right, a right side of the smartphone 50 may vibrate, and in a case of moving the camera to the left, a left side of the smartphone 50 may vibrate. Alternatively, in a case of moving the camera 18 to the right, the smartphone 50 may vibrate only once, and in a case of moving the camera to the left, the smartphone 50 may vibrate twice in succession.

### (Technical Effect)

Next, a description will be given of a technical effect obtained by the information processing system 10 according to the fifth example embodiment.

As described in FIG. 9, in the information processing system 10 according to the fifth example embodiment, the first guide information is displayed as information indicating in which direction the image capturing range of the image capturing unit 110 should be moved. In this way, it is possible to capture an image of the iris, properly, by the photographer following the guide to adjust the direction of the image capturing unit 110.

### Sixth Example Embodiment

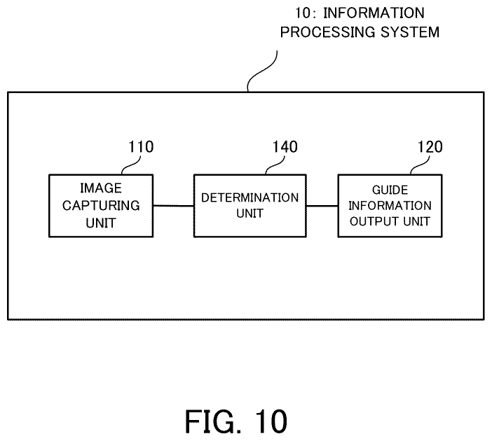

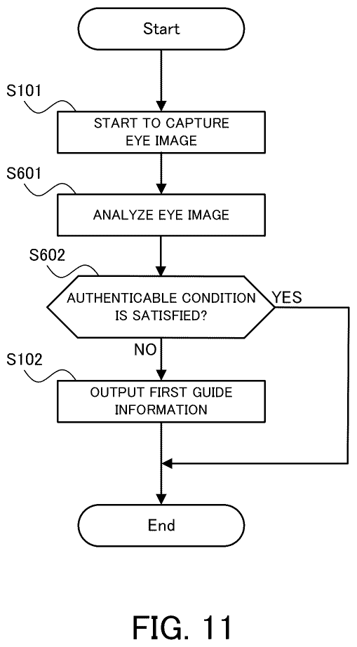

The information processing system 10 according to a sixth example embodiment will be described with reference to FIG. 10 and FIG. 11. The sixth example embodiment partially differs from the first to fifth example embodiments only in its configuration and operation, and may be the same as the first to fifth example embodiments in the other parts. For this reason, a part differing from each of the example embodiments described above will be described in detail below, and a description of the other overlapping parts will be omitted as appropriate.

### (Functional Configuration)

First, with reference to FIG. 10, a functional configuration of the information processing system 10 according to the sixth example embodiment will be described. FIG. 10 is a block diagram illustrating the functional configuration of the information processing system according to the sixth example embodiment. In FIG. 10, the same components as those illustrated in FIG. 2 carry the same reference numerals.

As illustrated in FIG. 10, the information processing system 10 according to the sixth example embodiment includes, as components for realizing the functions thereof, the image capturing unit 110, the guide information output unit 120, and a determination unit 140. That is, the information processing system 10 according to the sixth example embodiment includes the determination unit 140, in addition to the configuration in the first example embodiment (see FIG. 2). The determination unit 140 may be a processing block realized by the processor 11 (see FIG. 1), for example.

The determination unit 140 is configured to determine whether or not the eye image satisfies an authenticable condition, by analyzing the eye image. The “authenticable condition” is a condition set to determine whether or not the eye image allows the iris recognition. The determination unit 140 may determine that the eye image that satisfies the authenticable condition is the image that allows the iris recognition, and that the eye image that does not satisfy the authenticable condition is an image that does not allow the iris recognition.

The authenticable condition may include a plurality of conditions. In that case, it may be determined that the authenticable condition is satisfied when all of the plurality of conditions are satisfied, or it may be determined that the authenticable condition is satisfied when a predetermined number or more of the plurality of conditions are satisfied. The authenticable condition may include conditions relating to the position and size of the iris, and an eye-opening degree, for example. The authenticable condition may also include conditions relating to an image capturing environment (e.g., conditions relating to brightness and lighting reflection). Furthermore, the authenticable condition may also include conditions relating to camera parameters (e.g., resolution, sharpness, focus, exposure, shutter speed, etc.).

### (Flow of Operation)

Next, with reference to FIG. 11, a flow of operation of the information processing system 10 according to the sixth example embodiment will be described. FIG. 11 is a flowchart illustrating the flow of the operation of the information processing system according to the sixth example embodiment. In FIG. 11, the same steps as those illustrated in FIG. 3 carry the same reference numerals.

As illustrated in FIG. 11, when the operation of the information processing system 10 according to the sixth example embodiment is started, the image capturing unit 110 first starts to capture the eye image (step S101). Then, the determination unit 140 analyzes the captured eye image (step S601).

Then, the determination unit 140 determines whether or not the eye image satisfies the authenticable condition, based on an analysis result of the eye image (step S602). Here, when the eye image is determined not to satisfy the authenticable condition (the step S602: NO), the guide information output unit 120 outputs the first guide information (step S102).

On the other hand, when the eye image is determined to satisfy the authenticable condition (the step S602: YES), the step S102 is omitted. That is, the guide information output unit 120 does not output the first guide information.

### (Technical Effect)

Next, a description will be given of a technical effect obtained by the information processing system 10 according to the sixth example embodiment.

As described in FIG. 10 and FIG. 11, in the information processing system 10 according to the sixth example embodiment, it is determined whether or not the eye image satisfies the authenticable condition, and when the eye image does not satisfy the authenticable condition, the first guide information is outputted. In this way, it is possible to output appropriate first guide information, depending on a determination result of the eye image. In addition, since the first guide information is not outputted when the eye image satisfies the authenticable condition, it is possible to prevent the first guide information from being outputted even though the eye image of sufficient quality is captured.

### Seventh Example Embodiment

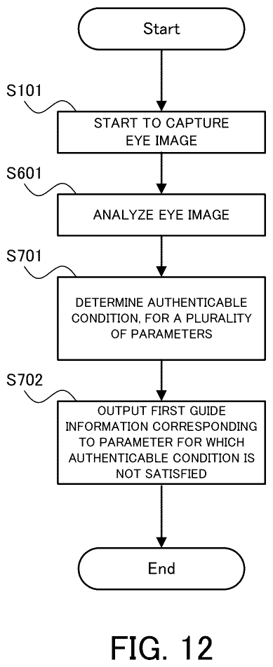

The information processing system 10 according to a seventh example embodiment will be described with reference to FIG. 12. The seventh example embodiment partially differs from the sixth example embodiment only in its operation, and may be the same as the first to sixth example embodiments in the other parts. For this reason, a part differing from each of the example embodiments described above will be described in detail below, and a description of the other overlapping parts will be omitted as appropriate.

### (Flow of Operation)

First, with reference to FIG. 12, a flow of operation of the information processing system 10 according to the seventh example embodiment will be described. FIG. 12 is a flowchart illustrating the flow of the operation of the information processing system according to the seventh example embodiment. In FIG. 12, the same steps as those illustrated in FIG. 11 carry the same reference numerals.

As illustrated in FIG. 12, when the operation of the information processing system 10 according to the seventh example embodiment is started, the image capturing unit 110 first starts to capture the eye image (step S101). Then, the determination unit 140 analyzes the captured eye image (step S601).

The determination unit 140 then determines whether or not the authenticable condition is satisfied, for each of a plurality of parameters, based on the analysis result of the eye image (step S701). For example, in a case where the authenticable condition is set for each of focus, brightness, resolution, and size, the determination unit 140 determines whether the authenticable condition is satisfied, for each of focus, brightness, resolution, and size.

After that, the guide information output unit 120 outputs the first guide information, based on the determination result for each parameter in the determination unit 140. Specifically, the guide information output unit 120 outputs the first guide information about a parameter for which it is determined that the authenticable condition is not satisfied (step S702). For example, when the authenticable condition is satisfied for focus and brightness, but the authenticable condition is not satisfied for resolution and size, the guide information output unit 120 outputs the first guide information about resolution and size. When the authenticable condition is not satisfied for all the parameters, the first guide information output unit 120 may output the first guide information about all the parameters. Furthermore, when the authenticable condition is satisfied for all the parameters, the first guide information output unit 120 may not output the first guide information.

### (Technical Effect)

Next, a description will be given of a technical effect obtained by the information processing system 10 according to the seventh example embodiment.

As described in FIG. 12, in the information processing system 10 according to the seventh example embodiment, the authenticable condition is determined for each of the plurality of parameters, and the first guide information is outputted based on the determination result. In this way, it is possible to output more appropriate first guide information, depending on each of the plurality of parameters.

### Eighth Example Embodiment

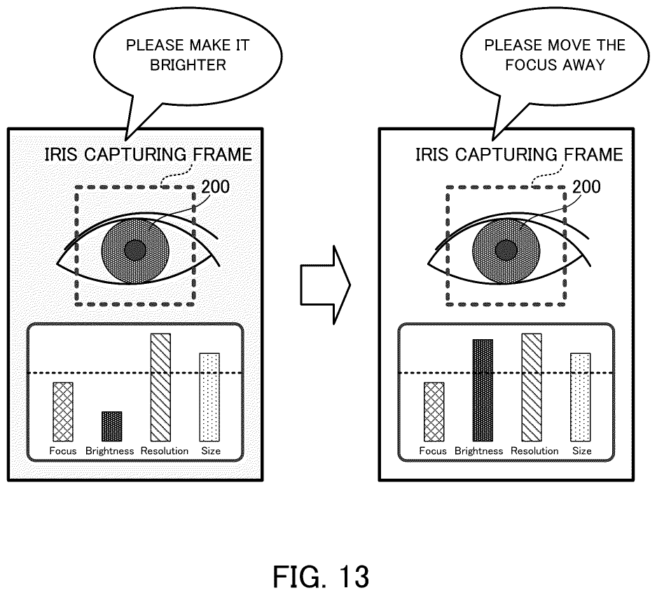

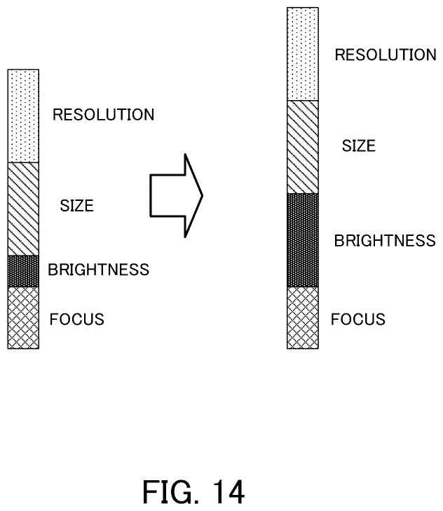

The information processing system 10 according to an eighth example embodiment will be described with reference to FIG. 13 and FIG. 14. The eighth example embodiment partially differs from the seventh example embodiment only in its operation, and may be the same as the first to seventh example embodiments in the other parts. For this reason, a part differing from each of the example embodiments described above will be described in detail below, and a description of the other overlapping parts will be omitted as appropriate.

### (Parameter Display)

First, with reference to FIG. 13, a description will be given of a display example of the first guide information by the information processing system 10 according to the eighth example embodiment. FIG. 13 is a plan view illustrating an output example of the first guide information in the information processing system according to the eighth example embodiment.

As illustrated in FIG. 13, the information processing system 10 according to the eighth example embodiment may display the determination result for each of the plurality of parameters by the determination unit 140, in a graph format on the display unit 130. The determination unit 140 may determine whether or not the authenticable condition is satisfied by determining whether or not each of the plurality of parameters exceeds a threshold (a dotted line in a graph) set in advance. In the example illustrated in FIG. 13, at an initial stage, the parameters for focus and brightness do not reach the threshold. Therefore, the determination unit 140 determines that the authenticable condition is not satisfied for focus and brightness. On the other hand, the parameters for resolution and size exceed the threshold. Therefore, the determination unit 140 determines that the authenticable condition is satisfied for resolution and size.

The determination unit 140 is further configured to determine a degree of deviation from the threshold (in other words, an unreached degree) for the parameter that does not yet reach the threshold. The guide information output unit 120 according to the present example embodiment is configured to output the first guide information in descending order from the parameter with the largest degree of deviation from the threshold. As already described, the parameters for focus and brightness do not yet reach the threshold at the initial stage, but the parameter for brightness has a larger degree of deviation from the threshold. In this case, the guide information output unit 120 first outputs the first guide information about brightness. For example, the guide information output unit 120 outputs the first guide information for outputting a message of “Please make it brighter” or “Please make it darker” (i.e., guide information for improving brightness). Then, after the parameter for brightness is improved, the guide information output unit 120 outputs the first guide information about focus. For example, the guide information output unit 120 outputs the first guide information for outputting a message of “Please move the focus away” or “Please move the focus closer” (i.e., guide information for improving focus).

### (Modification)

Next, with reference to FIG. 14, a description will be given of a modification of the first guide information displayed by the information processing system 10 according to the eighth example embodiment. FIG. 14 is a plan view illustrating the modification of the first guide information in the information processing system according to the eighth example embodiment.

As illustrated in FIG. 14, the information processing system 10 according to the eighth example embodiment may display the respective determination results for the parameters, as a single bar with the parameters connected vertically. The parameter improved by outputting the first guide information may be displayed such that a corresponding part is longer. In the example illustrated in FIG. 14, a part corresponding to brightness becomes longer due to an improvement in the parameter for brightness. Although given here is an example in which the length of the entire bar changes due to a change in each parameter, the length of the entire bar may be fixed, and a ratio of a part corresponding to each parameter may be changed.

### (Technical Effect)

Next, a description will be given of a technical effect obtained by the information processing system 10 according to the eighth example embodiment.

As described in FIG. 13 and FIG. 14, in the information processing system 10 according to the eighth example embodiment, the authenticable condition is determined based on the set threshold for each of the plurality of parameters, and the first guide information corresponding to each parameter is outputted in order corresponding to the degree of deviation from the threshold. In this way, it is possible to output pieces of the first guide information corresponding to the plurality of parameters in appropriate order. This allows the photographer to adjust the parameters in order of priority.

### Ninth Example Embodiment

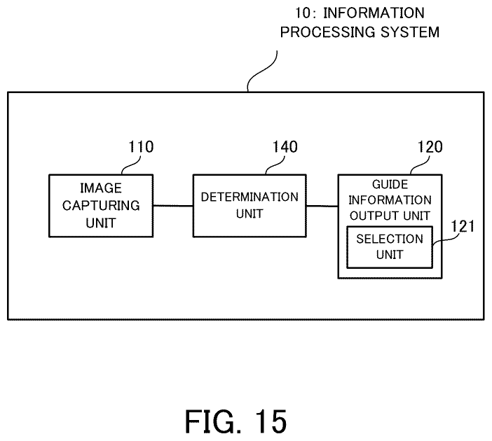

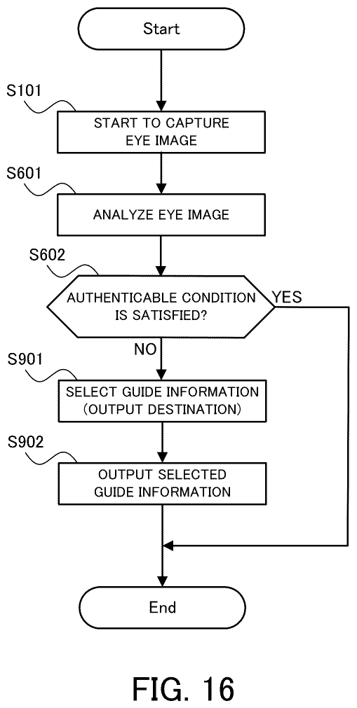

The information processing system 10 according to a ninth example embodiment will be described with reference to FIG. 15 and FIG. 16. The ninth example embodiment partially differs from the first to eighth example embodiments only in its configuration and operation, and may be the same as the first to eighth example embodiments in the other parts. For this reason, a part differing from each of the example embodiments described above will be described in detail below, and a description of the other overlapping parts will be omitted as appropriate.

### (Functional Configuration)

First, with reference to FIG. 15, a functional configuration of the information processing system 10 according to the ninth example embodiment will be described. FIG. 15 is a block diagram illustrating the functional configuration of the information processing system according to the ninth example embodiment. In FIG. 15, the same components as those illustrated in FIG. 10 carry the same reference numerals.

As illustrated in FIG. 15, the information processing system 10 according to the ninth example embodiment includes, as components for realizing the functions thereof, the image capturing unit 110, the guide information output unit 120, and the determination unit 140. In particular, the guide information output unit 120 according to the ninth example embodiment includes a selection unit 121. Furthermore, the guide information output unit 120 according to the ninth example embodiment is configured to output second guide information for the image capturing target, in addition to the first guide information for the photographer.

The selection unit 121 is configured to select at least one of the first guide information and the second guide information, as the guide information outputted by the guide information output unit 120. The selection unit 121 selects which guide information is to be outputted, based on the determination result for each of the plurality of parameters in the determination unit 140. For example, when the authenticable condition is not satisfied for a parameter requiring a response from the photographer (e.g., camera parameters), the selection unit 121 may select the first guide information for the photographer. Furthermore, when the authenticable condition is not satisfied for a parameter requiring a response from the image capturing target (e.g., the eye-opening degree), the selection unit 121 may select the second guide information for the image capturing target. In addition, when the authenticable condition is not satisfied for the parameter for which the response from both the photographer and the image capturing target is possible (e.g., the position of the iris), the selection unit 121 may select both the first guide information for the photographer and the second guide information for the image capturing target.

### (Flow of Operation)

Next, with reference to FIG. 16, a description will be given of a flow of operation of the information processing system 10 according to the ninth example embodiment. FIG. 16 is a flowchart illustrating the flow of the operation of the information processing system according to the ninth example embodiment. In FIG. 16, the same steps as those illustrated in FIG. 11 carry the same reference numerals.

As illustrated in FIG. 16, when the operation of the information processing system 10 according to the ninth example embodiment is started, the image capturing unit 110 first starts to capture the eye image (step S101). Then, the determination unit 140 analyzes the captured eye image (step S601).

Then, the determination unit 140 determines whether or not the eye image satisfies the authenticable condition, based on the analysis result of the eye image (step S602). Here, when the eye image is determined to satisfy the authenticable condition (the step S602: YES), the subsequent processes are omitted. That is, the guide information output unit 120 does not output the first guide information.

On the other hand, when the eye image is determined not to satisfy the authenticable condition (the step S602: NO), the selection unit 121 selects the guide information to be outputted, depending on the parameter that does not satisfy the authenticable condition (step S901). That is, it is determined whether to output the first guide information for the photographer, the second guide information for the image capturing target, or both the first guide information and the second guide information.

After that, the guide information output unit 120 outputs the guide information selected by the selection unit 121 (step S902).

### (Technical Effect)

Next, a description will be given of a technical effect obtained by the information processing system 10 according to the ninth example embodiment.

As described in FIG. 15 and FIG. 16, in the information processing system 10 according to the ninth example embodiment, the first guide information for the photographer and the second guide information for the image capturing target are selected and outputted as appropriate. In this way, it is possible to output appropriate guide information to an appropriate output destination, depending on a situation of the image capturing.

### 10th Example Embodiment

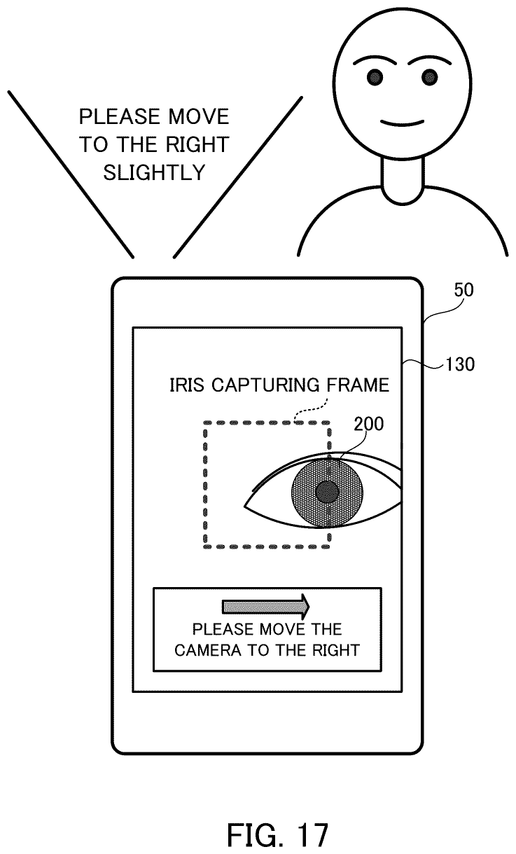

The information processing system 10 according to a 10th example embodiment will be described with reference to FIG. 17. The 10th example embodiment partially differs from the ninth example embodiment only in its operation, and may be the same as the first to the ninth example embodiments in the other parts. For this reason, a part differing from each of the example embodiments described above will be described in detail below, and a description of the other overlapping parts will be omitted as appropriate.

### (Output Aspect of Guide Information)

First, with reference to FIG. 17, a description will be given of an output aspect of the first guide information and the second guide information in the information processing system 10 according to the 10th example embodiment. FIG. 17 is a conceptual diagram illustrating an output example of the first guide information and the second guide information by the information processing system according to the 10th example embodiment.

As illustrated in FIG. 17, the information processing system 10 according to the 10th example embodiment is configured to output the first guide information for the photographer and the second guide information for the image capturing target in different aspects from each other. For example, the guide information output unit 120 displays the first guide information for the photographer on the display unit 130, while the guide information output unit 120 audio-outputs the second guide information for the image capturing target by using a speaker. In the example illustrated in FIG. 17, a message of “Please move the camera to the right” is displayed for the photographer. Meanwhile, a message of “Please move to the right slightly” is audio-outputted to the image capturing target.

FIG. 17 illustrates an example in which the first guide information is displayed as an image and the second guide information is audio-outputted, but this is just an example of a possible combination, and the combination of the output aspects is not particularly limited. For example, the first guide information may be outputted by using a vibration function, and the second guide information may be audio-outputted.

### (Technical Effect)

Next, a description will be given of a technical effect obtained by the information processing system 10 according to the 10th example embodiment.

As described in FIG. 17, in the information processing system 10 according to the 10th example embodiment, the first guide information and the second guide information are outputted in different aspects from each other. In this way, it is possible to output each of the first guide information for the photographer and the second guide information for the image capturing target, in a suitable manner. In the example illustrated in FIG. 17, the guide information indicating moving the camera (or the image capturing target himself moving) to the right is outputted to both the photographer and the image capturing target, but if the two types of guide information are outputted in a common aspect, an intention of the guide information may not be properly conveyed. For example, in a case where audio guidance of “Please move to the right” is outputted to both the photographer and the image capturing target, the photographer and the image capturing target may be unsure whether to move to the right as seen from their own perspective, or to the right as seen from the other person's perspective (i.e. to the left as seen from their own perspective). In the present example embodiment, however, the guidance information is outputted in a different aspect to the photographer and the image capturing target, and it is therefore possible to avoid the situation where the photographer and the image capturing target are unsure what to do as described above.

### 11th Example Embodiment

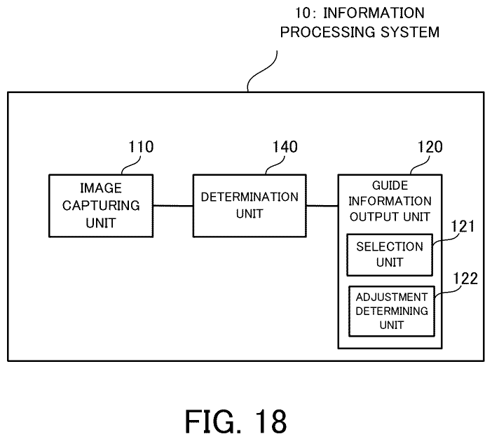

The information processing system 10 according to an 11th example embodiment will be described with reference to FIG. 18 to FIG. 20. The 11th example embodiment partially differs from the ninth and 10th example embodiments only in its operation, and may be the same as the first to 10th example embodiments in the other parts. For this reason, a part differing from each of the example embodiments described above will be described in detail below, and a description of the other overlapping parts will be omitted as appropriate.

### (Functional Configuration)

First, with reference to FIG. 18, a functional configuration of the information processing system 10 according to the 11th example embodiment will be described. FIG. 18 is a block diagram illustrating the functional configuration of the information processing system according to the 11th example embodiment. In FIG. 18, the same components as those illustrated in FIG. 15 carry the same reference numerals.

As illustrated in FIG. 18, the information processing system 10 according to the 11th example embodiment includes, as components for realizing the functions thereof, the image capturing unit 110, the guide information output unit 120, and the determination unit 140. In particular, the guide information output unit 120 according to the 11th example embodiment includes the selection unit 121 and an adjustment determining unit 122. That is, the information processing system 10 according to the ninth example embodiment includes the adjustment determining unit 122. in addition to the configuration in the ninth example embodiment (see FIG. 15).

The adjustment determining unit 122 is configured to determine whether or not the first guide information to be outputted (or the guide information to be already outputted) by the guide information output unit 120 includes information requiring the photographer to make adjustment. For example, in a case where the first guide information is information about camera parameters, the photographer is required to adjust the camera parameters. In this case, the adjustment determining unit 122 may determine that the first guide information includes the information requiring the adjustment.

The guide information output unit 120 according to the present example embodiment is configured to output the second guide information to the image capturing target, based on a determination result by the adjustment determining unit 122. Specifically, when the adjustment determining unit 122 determines that the photographer is required to make adjustment, the guide information output unit 120 outputs, to the image capturing target, the second guide information instructing the image capturing target to maintain its posture. The second guide information is outputted to prevent the image of the iris from being blurred as the image capturing target moves.

As can be seen from the above example, the “adjustment” to be determined in the present example embodiment only takes a relatively short time. Therefore, in a case where the adjustment takes a long time, the second guide information described in the present example embodiment (i.e., the guide information for maintaining the posture of the photographer) mays not be outputted. A response when the adjustment takes a long time will be described later in a 12th example embodiment.

### (Adjustment Determining Operation)

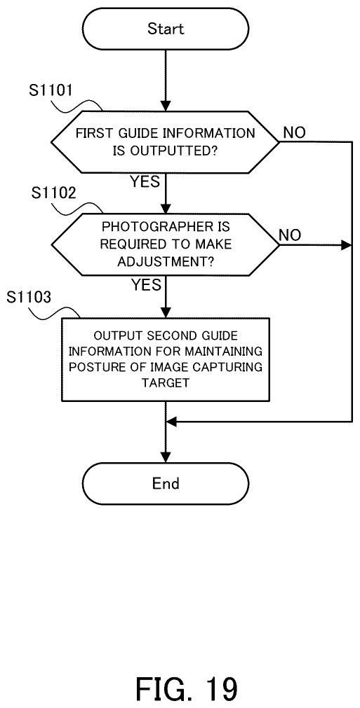

Next, with reference to FIG. 19, a description will be given of a flow of an adjustment determining operation (i.e., an operation of the adjustment determining unit 122) in the information processing system 10 according to the 11th example embodiment. FIG. 19 is a flowchart illustrating the flow of the adjustment determining operation by the information processing system according to the 11th example embodiment.

As illustrated in FIG. 19, the adjustment determining unit 122 of the information processing system 10 according to the 11th example embodiment first determines whether or not the first guide information is outputted to the photographer (step S1101). When the first guide information is not outputted (the step S1101: NO), the subsequent steps may be omitted.

When the first guide information is outputted (the step S1101: YES), the adjustment determining unit 122 determines whether or not the outputted first guide information requires the photographer to make adjustment (step S1102). When the first guide information does not require the photographer to make adjustment (the step S1102: NO), the subsequent steps may be omitted.

When the first guide information requires the photographer to make adjustment (the step S1102: YES), the guide information output unit 120 outputs, to the image capturing target, the second guide information instructing the image capturing target to maintain its posture (step S1103). The guide information output unit 120 may also output information indicating that the image capturing target may be released from maintaining the posture, after the image capturing is ended.

### (Output Example of Guide Information)

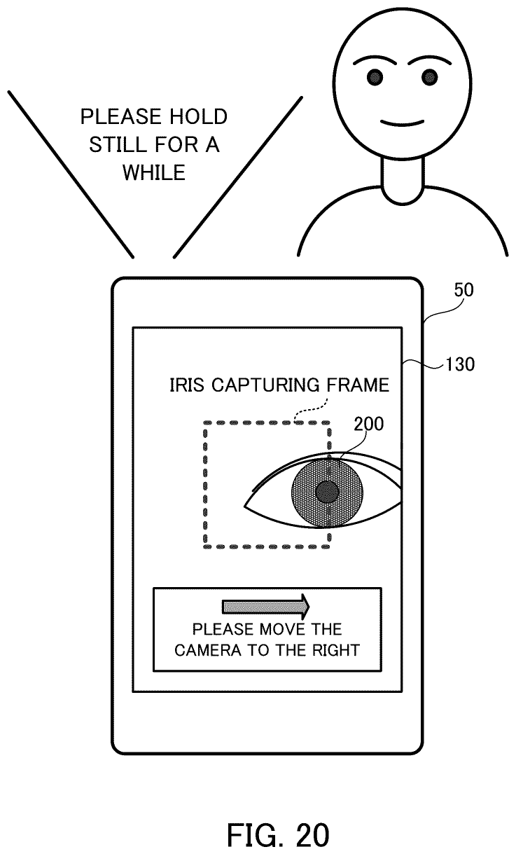

Next, with reference to FIG. 20, a description will be given of an output example of the guide information by the information processing system 10 according to the 11th example embodiment. FIG. 20 is a conceptual diagram illustrating an output example of the first guide information and the second guide information by the information processing system according to the 11th example embodiment.

As illustrated in FIG. 20, let us assume that the first guide information is intended to display a message of “Please move the camera to the right”. In this case, the photographer is required to move the camera 18 to the right to set the iris 200 of the image capturing target in an appropriate position (i.e., to include the iris 200 within the iris capturing frame). Then, the photographer is expected to capture the eye image of the image capturing target, after the adjustment of the camera 18.

In such a case, the adjustment determining unit 122 determines that the first guide information requires the photographer to make adjustment. The guide information output unit 120 then audio-outputs a message of “Please hold still for a while” to the image capturing target. This allows the posture of the image capturing target to be maintained, and the photographer having finished the adjustment, is able to capture an image with reduced blur.

The second guide information may also include information other than the information for maintaining the posture. For example, the second guide information may include the content of the first guide information in order to inform the image capturing target what type of adjustment the photographer is making. More specifically, the second guide information may be intended to output, to the image capturing target, a message of “The photographer is currently adjusting the position of the camera. Please hold till for a while.”

### (Technical Effect)

Next, a description will be given of a technical effect obtained by the information processing system 10 according to the 11th example embodiment.

As described in FIG. 18 to FIG. 20, in the information processing system 10 according to the 11th example embodiment, when the first guide information requiring the photographer to make adjustment is outputted, the second guide information instructing the image capturing target to maintain its posture is outputted to the image capturing target. In this way, since the image capturing target is instructed not to move, the photographer having finished the adjustment based on the first guide information is able to properly capture the iris if the image capturing target.

### 12th Example Embodiment

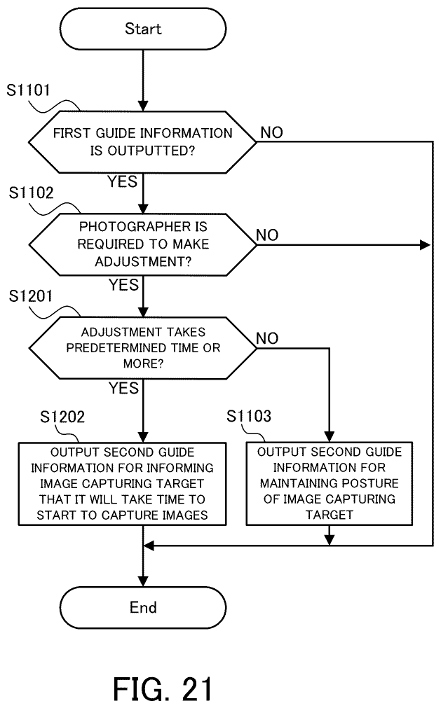

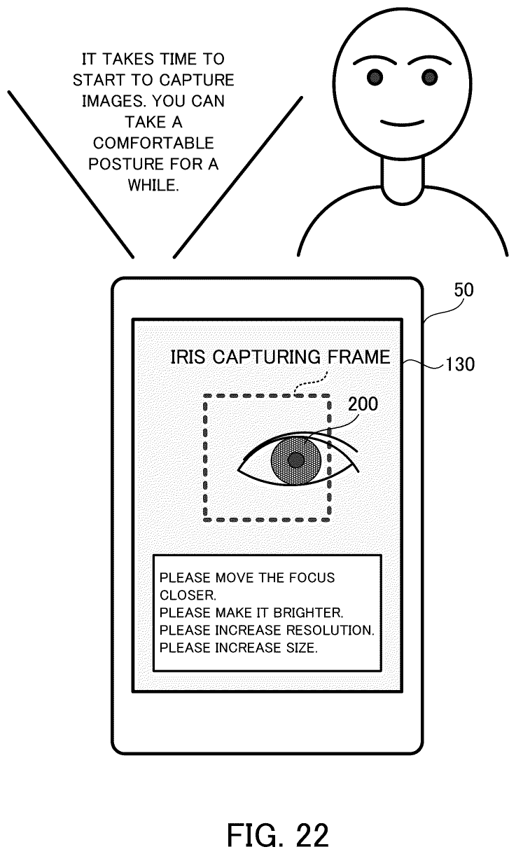

The information processing system 10 according to a 12th example embodiment will be described with reference to FIG. 21 and FIG. 22. The 12th example embodiment partially differs from the 11th example embodiment only in its operation, and may be the same as the first to 11th example embodiments in the other parts. For this reason, a part differing from each of the example embodiments described above will be described in detail below, and a description of the other overlapping parts will be omitted as appropriate.

### (Adjustment Determining Operation)

First, with reference to FIG. 21, a flow of the adjustment determining operation in the information processing system 10 according to the 12th example embodiment will be described. FIG. 21 is a flowchart illustrating the flow of the adjustment determining operation by the information processing system according to the 12th example embodiment. In FIG. 21, the same steps as those illustrated in FIG. 19 carry the same reference numerals.

As illustrated in FIG. 21, the adjustment determining unit 122 of the information processing system 10 according to the 12th example embodiment first determines whether or not the first guide information is outputted to the photographer (step S1101). When the first guide information is not outputted (the step S1101: NO), the subsequent steps may be omitted.

When the first guide information is outputted (the step S1101: YES), the adjustment determining unit 122 determines whether or not the outputted first guide information requires the photographer to make adjustment (step S1102). When the first guide information does not require the photographer to make adjustment (the step S1102: NO), the subsequent steps may be omitted.

When the first guide information requires the photographer to make adjustment (the step S1102: YES), the adjustment determining unit 122 determines whether or not the adjustment that the photographer is required takes a predetermined time or more (step S1201). The “predetermined time” here is a threshold for determining whether or not the adjustment takes a relatively long time. The predetermined time may be set based on a time in which the image capturing target can maintain the posture without feeling burdened, for example.

When it is determined that the adjustment takes the predetermined time or more (the step S1201: YES), the guide information output unit 120 outputs, to the image capturing target, the second guide information for informing the image capturing target that it takes time to start to capture images (step S1202). On the other hand, when it is determined that the adjustment does not take the predetermined time or more (the step S1201: NO), the guide information output unit 120 may output, to the image capturing target, the second guide information for maintaining the posture (step S1103). That is, the guide information output unit 120 may output the same second guide information as that described in the 11th example embodiment.

### (Output Example of Guide Information)

Next, with reference to FIG. 22, a description will be given of an output example of the guide information by the information processing system 10 according to the 12th example embodiment. FIG. 22 is a conceptual diagram illustrating an output example of the first guide information and the second guide information by the information processing system according to the 12th example embodiment.

As illustrated in FIG. 22, the first guide information may include the guide information about the plurality of parameters. For example, the guide information for improving focus may be guide information for outputting a message of “Please move the focus closer” or “Please move the focus away”. The guide information for improving brightness may be guide information for outputting a message of “Please make it brighter” or “Please make it darker”. The guide information for improving resolution may be guide information for outputting a message of “Please increase the resolution” or “Please reduce the resolution”. The guide information for improving size may be guide information for outputting a message of “Please increase the size” or “Please reduce the size”. When the plurality of messages are displayed in this way, the photographer is required to make a plurality of types of adjustments in order to adjust each parameter. Then, the photographer is expected to capture the eye image of the image capturing target, after it takes a considerable amount of time to complete all of the plurality of types of adjustment and then capture an image of the target.

In such a case, the adjustment determining unit 122 determines that the first guide information requires a predetermined time or more of adjustment. Then, the guide information output unit 120 audio-outputs, to the image capturing target, a message of “It takes time to start to capture images. You can take a comfortable posture for a while.” This enables the image capturing target to wait for the image capturing in a relaxed state.

When the second guide information described above is outputted, the guide information output unit 120 may newly output the second guide information for informing the image capturing target that the image capturing is to be performed soon, after a lapse of a short period of time. For example, the guide information output unit 120 may output a message of “The adjustment will be finished soon. Please hold still for a while.”

### (Technical Effect)

Next, a description will be given of a technical effect obtained by the information processing system 10 according to the 12th example embodiment.

As described in FIG. 21 and FIG. 22, in the information processing system 10 according to the 12th example embodiment, when the first guide information requiring a predetermined time or more of adjustment is outputted to the photographer, the second guide information indicating that it takes time to start to capture images, is outputted to the image capturing target. In this way, when it takes time to start to capture images due to the adjustment by the photographer, a burden on the image capturing target may be reduced.

### 13th Example Embodiment

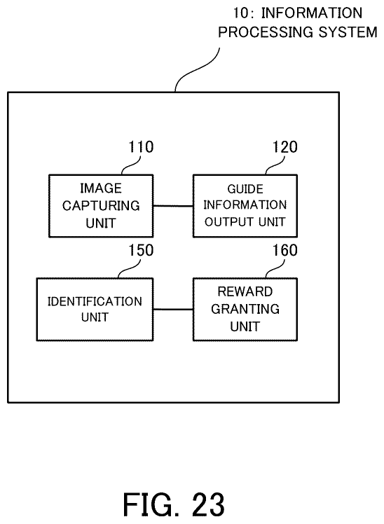

The information processing system 10 according to a 13th example embodiment will be described with reference to FIG. 23 to FIG. 25. The 13th example embodiment partially differs from the first to 12th example embodiments only in its configuration and operation, and may be the same as the first to 12th example embodiments in the other parts. For this reason, a part differing from each of the example embodiments described above will be described in detail below, and a description of the other overlapping parts will be omitted as appropriate.

### (Functional Configuration)

First, with reference to FIG. 23, a functional configuration of the information processing system 10 according to the 13th example embodiment will be described. FIG. 23 is a block diagram illustrating the functional configuration of the information processing system according to the 13th example embodiment. In FIG. 23, the same components as those illustrated in FIG. 2 carry the same reference numerals.

As illustrated in FIG. 23, the information processing system 10 according to the 13th example embodiment includes, as components for realizing the functions thereof, the image capturing unit 110, the guide information output unit 120, an identification unit 150, and a reward granting unit 160. That is, the information processing system 10 according to the 13th example embodiment further includes the identification unit 150 and the reward granting unit 160, in addition to the configuration in the first example embodiment (see FIG. 2). Each of the identification unit 150 and the reward granting unit 160 may be a processing block realized by the processor 11 (see FIG. 1), for example.

The identification unit 150 is configured to identify an image capturing cooperator located near the image capturing target. Here, the term “image capturing cooperator” refers to a user expressing a willingness to cooperate with the image capturing. The identification unit 150 may acquire position information from each of a plurality of image capturing cooperators registered in advance, and may identify the image capturing cooperator located within a predetermined distance from the image capturing target, for example. The image capturing cooperator identified by the identification unit 150 is located near the image capturing target, and it may be thus determined that he/she is an image capturing cooperator who can actually be asked to capture images. A request for the identified image capturing cooperator to capture images, may be made by the image capturing target, or may be made automatically by the system (i.e., matching may be automatically performed). A configuration for matching with the image capturing cooperator will be described in detail later in another example embodiment.

The reward granting unit 160 is configured to grant a reward for capturing the eye image, to the image capturing cooperator authenticated by using the image capturing unit 110 carried/owned by the image capturing target. The image capturing cooperator asked to capture images, picks up the terminal carried/owned by the image capturing target, in order to capture the eye image of the image capturing target by using the image capturing unit 110 carried/owned by the image capturing target. Therefore, it may be presumed that the image capturing cooperator authenticated by using the image capturing unit 110 carried/owned by the image capturing target is the image capturing cooperator actually asked to capture images. Therefore, the reward granting unit 160 grants a reward to the authenticated image capturing cooperator.

The authentication using the image capturing unit may be face authentication using a face image, for example. This authentication processing may be performed by the information processing system 10, or may be performed by an authentication apparatus external to the system. In a case where the face authentication is performed, an image of a face of the image capturing cooperator who captures the eye image is captured by the image capturing unit 110, and that face image is collated/verified with a face image of the image capturing cooperator identified by the identification unit 150 to perform the authentication. The image to be used for the face authentication may be obtained by capturing the face of the image capturing cooperator with a front camera of the smartphone 50 when the image capturing cooperator captures images of the image capturing target with a rear camera of the smartphone 50. An authentication method is not limited to the face authentication, and the iris recognition, fingerprint authentication, or the like, may also be performed.

### (Cooperator Identifying Operation)

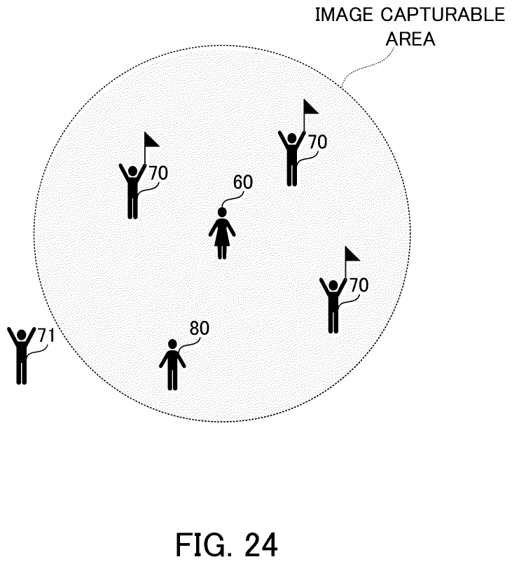

Next, with reference to FIG. 24, a more specific description will be given of a cooperator identifying operation (i.e., an operation of the identification unit 150) by the information processing system 10 according to the 13th example embodiment. FIG. 24 is a conceptual diagram illustrating the cooperator identifying operation by the information processing system according to the 13th example embodiment.

As illustrated in FIG. 24, the identification unit 150 identifies the image capturing cooperator located in an image capturable area set near an image capturing target 60. In the example illustrated in FIG. 24, image capturing cooperators 70 located in the image capturable area are displayed as icons with the hands raised and holding flags. An image capturing cooperator 71 located outside the image capturable area is displayed as an icon with the hands raised, but not holding a flag. A user 80 other than the image capturing cooperators is displayed as an icon with the hands down. The identification unit 150 may display such an image on the display 130.

### (Flow of Operation)

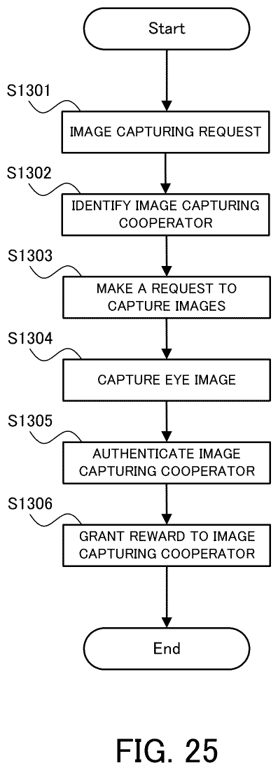

Next, with reference to FIG. 25, a description will be given of a flow of operation of the information processing system 10 according to the 13th example embodiment. FIG. 25 is a flowchart illustrating the flow of the operation of the information processing system according to the 13th example embodiment.

As illustrated in FIG. 25, in the information processing system 10 according to the 13th example embodiment, the image capturing target first inputs an image capturing request to the information processing system 10 (step S1301). When the image capturing request is inputted, the identification unit 150 identifies the image capturing cooperator located near the image capturing target (step S1301).

Then, the image capturing target asks the identified image capturing cooperator to capture images (step S1303). Then, the image capturing cooperator asked to capture images, captures the eye image of the image capturing target (step S1304).

Then, the information processing system 10 performs authentication on the image capturing cooperator having captured the eye image (step S1305). Then, the reward granting unit 160 grants a reward for capturing the eye image to the authenticated image capturing cooperator (step S1306).

The reward granting unit 160 may grant the reward as points or electronic money. The reward granted by the reward granting unit 160 may have a fixed amount or a variable amount depending on the situation. For example, the reward granting unit 160 may increase the reward when the image capturing progresses smoothly or when the captured images have high quality. Alternatively, the reward granting unit 160 may increase the reward, for the image capturing cooperator located far from the image capturing target when identified (i.e., the image capturing cooperator coming all the way from a distant location to capture images).

### (Technical Effect)

Next, a description will be given of a technical effect obtained by the information processing system 10 according to the 13th example embodiment.

As described in FIG. 23 to FIG. 25, in the information processing system 10 according to the 13th example embodiment, the reward for capturing images is given to the image capturing cooperator authenticated by using the image capturing unit of the image capturing target. In this way, the image capturing target may ask the image capturing cooperator located near the photographer to capture images of the iris. In addition, by paying a reward to the image capturing cooperator, it is possible to encourage active cooperation in capturing images.

### 14th Example Embodiment

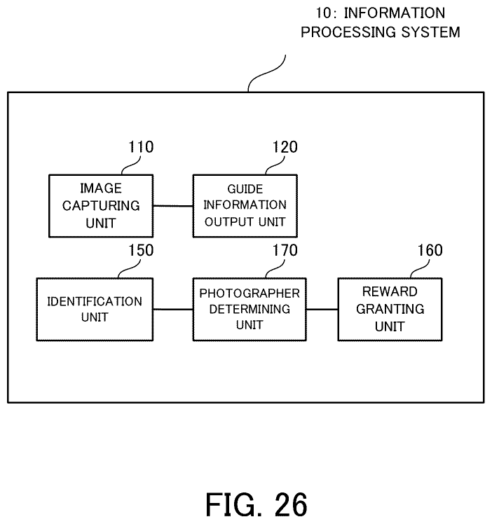

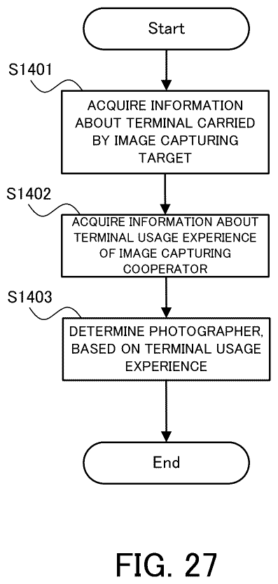

The information processing system 10 according to a 14th example embodiment will be described with reference to FIG. 26 and FIG. 27. The 14th example embodiment partially differs from the 13th example embodiment only in its components and operation, and may be the same as the first to 13th example embodiments in the other parts. For this reason, a part differing from each of the example embodiments described above will be described in detail below, and a description of the other overlapping parts will be omitted as appropriate.

### (Functional Configuration)

First, with reference to FIG. 26, a description will be given of a functional configuration of the information processing system 10 according to the 14th example embodiment. FIG. 26 is a block diagram illustrating the functional configuration of the information processing system according to the 14th example embodiment. In FIG. 26, the same components as those illustrated in FIG. 23 carry the same reference numerals.

As illustrated in FIG. 26, the information processing system 10 according to the 14th example embodiment includes, as components for realizing the functions thereof, the image capturing unit 110, the guide information output unit 120, the identification unit 150, the reward granting unit 160, and a photographer determining unit 170. That is, the information processing system 10 according to the 14th example embodiment further includes the photographer determining unit 170, in addition to the configuration in the 13th example embodiment (see FIG. 23). The photographer determining unit 170 may be a processing block realized by the processor 11 (see FIG. 1), for example.

The photographer determining unit 170 is configured to determine the photographer who will actually capture the eye image, from among the plurality of image capturing cooperators identified by the identification unit 150. That is, the photographer determining unit 170 has a function for automatically matching the image capturing target and the image capturing cooperator. Especially, the photographer determining unit 170 according to the present example embodiment preferentially determines that the image capturing cooperator having experience of using the image capturing unit 110 (i.e., the camera 18) carried/owned by the image capturing target is the photographer, from among the plurality of identified image capturing cooperators. Information about the experience using the camera 18 may be registered in advance in association with information about the photographer.

### (Cooperator Determining Operation)

Next, with reference to FIG. 27, a description will be given of a flow of a cooperator determining operation (i.e., an operation of the photographer determining unit 170 described above) by the information processing system 10 according to the 14th example embodiment. FIG. 27 is a flowchart illustrating the flow of the photographer determining operation using the information processing system in the 14th example embodiment.

As illustrated in FIG. 27, the photographer determining unit 170 according to the 14th example embodiment first acquires information about the terminal carried/owned by the image capturing target (step S1401). For example, the photographer determining unit 170 acquires information for identifying a model, from the smartphone 50 equipped with the camera 18 that captures the eye image.

Then, the photographer determining unit 170 acquires information about terminal usage experience, from each of the plurality of image capturing cooperators (step S1402). That is, it acquires information for identifying the terminals that the image capturing cooperators having used before.

Then, the photographer determining unit 170 determines the photographer to be asked to capture the eye image, based on the information about terminal usage experience acquired from the image capturing cooperators (step S1403). Specifically, it determines whether or not the terminal carried/owned by the image capturing target is included in the information about terminal usage experience of the image capturing cooperators, and preferentially determines that the image capturing cooperator with usage experience is the photographer. In a case where there are plurality of image capturing cooperators with usage experience, the image capturing cooperator with more usage experience may be preferentially determined to be the photographer. Alternatively, the photographer may be determined by using another condition that differs from usage experience. For example, the image capturing cooperator located close to the photographer, or the image capturing cooperator previously having high evaluation, may be preferentially determined to be the photographer.

After the photographer is determined, the photographer determining unit 170 may transmit the image capturing request to the image capturing cooperator determined to be the photographer. In this case, the photographer determining unit 170 may transmit the image capturing request after confirming with the image capturing target whether the image capturing request may be made. At the time of confirmation, the image capturing target may be able to manually change the photographer. On the other hand, the image capturing cooperator having received the image capturing request may be able to select whether or not to accept the image capturing request. In a case where the image capturing cooperator does not accept the image capturing request, the photographer determining unit 170 may perform processing of determining a new photographer from among the other image capturing cooperators.

### (Technical Effect)

Next, a description will be given of a technical effect obtained by the information processing system 10 according to the 14th example embodiment.

As described in FIG. 26 and FIG. 27, in the information processing system 10 according to the 14th example embodiment, at the time of determining the photographer who will actually capture the eye image from among the plurality of image capturing cooperators, the experience of using the image capturing unit 110 (in other words, the camera 18) of the terminal carried/owned by the image capturing target is taken into consideration. In this way, since the image capturing cooperator with usage experience may be preferentially asked to capture images, it is possible to capture the eye image, more properly.

### 15th Example Embodiment

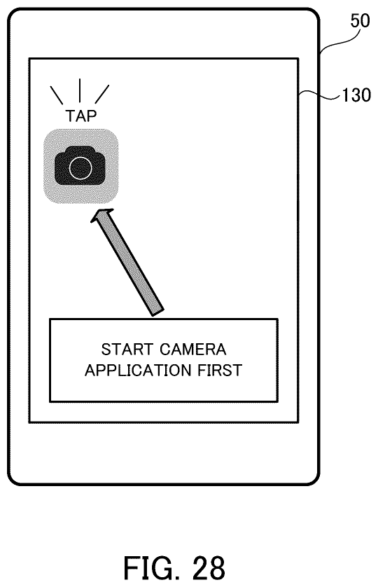

The information processing system 10 according to a 15th example embodiment will be described with reference to FIG. 28. The 15th example embodiment partially differs from the 13th and 14th example embodiments only in its components and operation, and may be the same as the first to 14th example embodiments in the other parts. For this reason, a part differing from each of the example embodiments described above will be described in detail below, and a description of the other overlapping parts will be omitted as appropriate.

### (Output Example of Guide Information)

First, with reference to FIG. 28, a description will be given of the first guide information outputted by the information processing system 10 according to the 15th example embodiment. FIG. 28 is a plan view illustrating a display example of the first guide information by the information processing system according to the 15th example embodiment

As illustrated in FIG. 28, in the information processing system 10 according to the 15th example embodiment, the guide information output unit 120 outputs the first guide information to the image capturing cooperator who captures the eye image, in order to make the image capturing unit 110 carried/owned by the image capturing target, ready for the image capturing. For example, the first guide information may be to display a message of “Please start the camera application first” as illustrated in the drawing. The first guide information may also indicate a position of the camera application with an arrow, or may display an action (e.g., tap) to start the camera application.

The guide information acquisition unit 120 may not output the first guide information in a case where the image capturing cooperator has experience of using the image capturing unit 110 carried/owned by the image capturing target. That is, in a case where it is clear that the image capturing cooperator knows how to make the image capturing unit 110 ready for the image capturing, the output of the first guide information described above may be omitted.

### (Technical Effect)

Next, a description will be given of a technical effect obtained by the information processing system 10 according to the 15th example embodiment.

As described in FIG. 28, in the information processing system 10 according to the 15th example embodiment, the first guide information is outputted to make the image capturing unit 110 ready for the image capturing. In this way, smooth image capturing is possible even in a situation where the photographer uses the image capturing unit 110 of the image capturing target to capture images (in other words, a situation where the photographer uses another person's camera 18 to capture images).

### 16th Example Embodiment

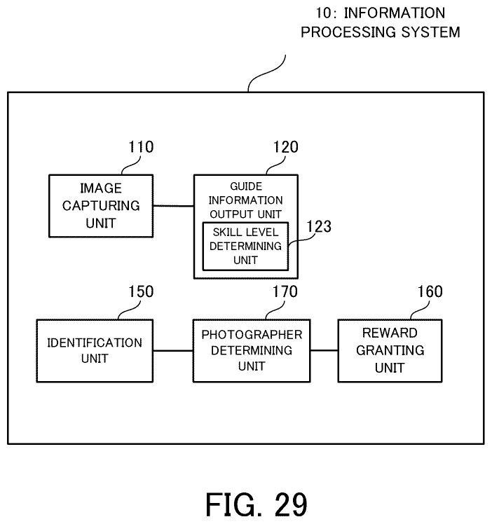

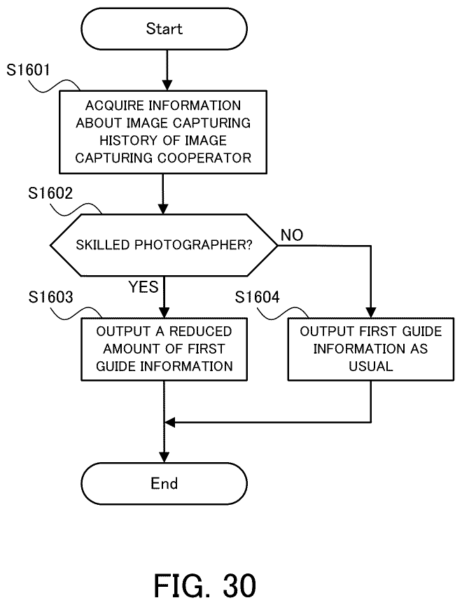

The information processing system 10 according to a 16th example embodiment will be described with reference to FIG. 29 and FIG. 30. The 16th example embodiment partially differs from the 13th to 15th example embodiments only in its components and operation, and may be the same as the first to 15th example embodiments in the other parts. For this reason, a part differing from each of the example embodiments described above will be described in detail below, and a description of the other overlapping parts will be omitted as appropriate.

### (Functional Configuration)

First, with reference to FIG. 29, a functional configuration of the information processing system 10 according to the 16th example embodiment will be described. FIG. 29 is a block diagram illustrating the functional configuration of the information processing system according to the 16th example embodiment. In FIG. 29, the same components as those illustrated in FIG. 26 carry the same reference numerals.

As illustrated in FIG. 29, the information processing system 10 according to the 16th example embodiment includes, as components for realizing the functions thereof, the image capturing unit 110, the guide information output unit 120, the identification unit 150, the reward granting unit 160, and the photographer determining unit 170. In particular, the guide information output unit 120 according to the 16th example embodiment includes a skill level determining unit 123.

The skill level determining unit 123 is configured to determine whether or not the image capturing cooperator who captures the eye image is a skilled photographer with a lot of experience of capturing the eye image. For example, the skill level determining unit 123 may determine that the image capturing cooperator previously having captured the eye image more than a predetermined number of times is a skilled photographer. Alternatively, the skill level determining unit 123 may determine that the image capturing cooperator previously having captured the eye image with overall high quality (e.g., an average value of the image quality exceeds a specified value) is a skilled photographer.

### (Skill Level Determining Operation)

Next, with reference to FIG. 30, a description will be given of a flow of a skill level determining operation (i.e., an operation of the skill level determining unit 123 described above) by the information processing system 10 according to the 16th example embodiment. FIG. 30 is a flowchart illustrating the flow of the skill level determining operation by the information processing system according to the 16th example embodiment.

As illustrated in FIG. 30, the skill level determining unit 123 according to the 16th example embodiment first acquires information about an image capturing history of the image capturing cooperator (step S1601). The image capturing history information may include information about the number of times to previously capture the eye image and the quality of the eye image previously captured.

Then, the skill level determining unit 123 determines whether or not the image capturing cooperator is a skilled photographer, based on the acquired image capturing history information (step S1602). When the image capturing cooperator is a skilled photographer (the step S1602: YES), the guide information output unit 120 outputs a smaller amount of the first guide information than usual (step S1603). On the other hand, when the image capturing cooperator is not a skilled photographer (the step S1602: NO), the guide information output unit 120 outputs the first guide information as usual (step S1604).

When reducing the amount of the first guide information, the guide information output unit 120 may reduce the types of the first guide information to be outputted. Alternatively, the guide information output unit 120 may reduce the number of times to output the first guide information. In addition, the guide information output unit 120 may increase an amount of the second guide information when reducing the amount of the first guide information. That is, while reducing the amount of the guide information to be outputted to the photographer, the guide information output unit 120 may increase the amount of the guide information to be outputted to the image capturing target.

### (Technical Effect)

Next, a description will be given of a technical effect obtained by the information processing system 10 according to the 16th example embodiment.

As described in FIG. 29 and FIG. 30, in the information processing system 10 according to the 16th example embodiment, when the image capturing cooperator is a skilled photographer, a reduced amount of first guide information is outputted. In this way, it is possible to output an appropriate amount of first guide information, depending on a skill level of the image capturing cooperator. Specifically, it is possible to prevent the first guide information, which is unnecessary for a skilled photographer with a lot of experience of capturing images (e.g., information that is obvious to a skilled photographer), from being outputted.

### 17th Example Embodiment

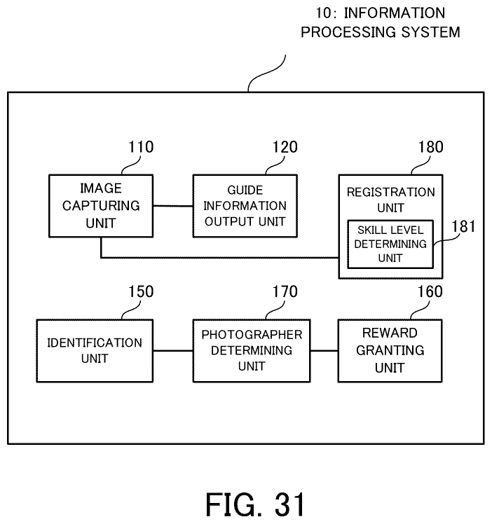

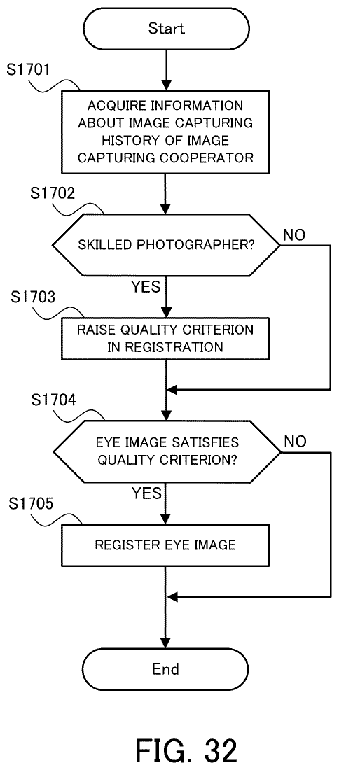

The information processing system 10 according to a 17th example embodiment will be described with reference to FIG. 31 and FIG. 32. The 17th example embodiment partially differs from the 13th to 16th example embodiments only in its components and operation, and may be the same as the first to 16th example embodiments in the other parts. For this reason, a part differing from each of the example embodiments described above will be described in detail below, and a description of the other overlapping parts will be omitted as appropriate.

### (Functional Configuration)

First, with reference to FIG. 31, a functional configuration of the information processing system 10 according to the 17th example embodiment will be described. FIG. 31 is a block diagram illustrating the functional configuration of the information processing system according to the 17th example embodiment. In FIG. 31, the same components as those illustrated in FIG. 26 carry the same reference numerals.

As illustrated in FIG. 31, the information processing system 10 according to the 16th example embodiment includes, as components for realizing the functions thereof, the image capturing unit 110, the guide information output unit 120, the identification unit 150, the reward granting unit 160, a photographer determining unit 170, and a registration unit 180. That is, the information processing system 10 according to the 16th example embodiment further includes the registration unit 180, in addition to the configuration in the 14th example embodiment (see FIG. 26). The registration unit 180 may be a processing block realized by the processor 11 (see FIG. 1), for example.

The registration unit 180 is configured to registered the eye image of the image capturing target captured by the image capturing unit 110, as a registration image to be used for the iris recognition. The registration unit 180 may be configured to store the registration image in the storage apparatus 14 (see FIG. 1), for example. The registration unit 180 registers the eye image as the registration image, when the captured eye image satisfies a predetermined quality criterion. The “predetermined quality criterion” is a criterion set to determine whether or not the eye image has sufficient quality to be used as the registration image for the iris recognition. Therefore, the registration unit 180 does not register the eye image of low quality as the registration image.

The registration unit 180 includes a skill level determining unit 181. The skill level determining unit 181 may have functions that are the same as or similar to those of the skill level determining unit 123 described in the 16th example embodiment. That is, the skill level determining unit 181 is configured to determine whether or not the image capturing cooperator who captures the eye image is a skilled photographer with a lot of experience of capturing the eye image.

### (Image Registering Operation)

Next, with reference to FIG. 32, a description will be given of a flow of an image registering operation (i.e., an operation of the registration unit 180 described above) by the information processing system 10 according to the 17th example embodiment. FIG. 32 is a flowchart illustrating the flow of the image registering operation by the information processing system according to the 17th example embodiment.

As illustrated in FIG. 32, the registration unit 180 according to the 17th example embodiment first acquires the image capturing history information from the image capturing cooperator (step S1701). The image capturing history information may include information about the number of times to previously capture the eye image and the quality of the eye image previously captured.

Then, the skill level determining unit 181 determines whether or not the image capturing cooperator is a skilled photographer, based on the acquired image capturing history information (step S1702). When the image capturing cooperator is a skilled photographer (the step S1702: YES), the registration unit 180 raises the quality criterion at the time of registering the eye image (step S1703). That is, it ensures that only the image of higher quality than usual is registered as the registration image. In a case where the skill level of a skilled photographer is known, an increment of the quality criterion may be change depending on the skill level. On the other hand, when the photographer is not a skilled photographer (the step S1702: NO), above-mentioned step S1703 is omitted. That is, the predetermined quality criterion remains the same as usual.

After that, the registration unit 180 determines whether or not the captured eye image satisfies the predetermined quality criterion (step S1704). When the eye image satisfies the predetermined quality criterion (the step S1704: YES), the registration unit 180 registers the eye image as the registration image (step S1705). When the image capturing cooperator is a skilled photographer, the predetermined quality criterion is raised. Therefore, the eye image captured by a skilled photographer is hardly registered as the registration image. In the case of a skilled photographer, however, the quality of the captured eye image tends to be high. Thus, even raising the predetermined quality criterion does not cause a problem that would prevent successful registration.

On the other hand, when the eye image does not satisfy the predetermined quality criterion (the step S1704: NO), the step S1705 described above is omitted. That is, the registration unit 180 does not register the eye image as the registration image. In this case, the registration unit 180 may output an instruction to capture a new eye image.

### (Technical Effect)

Next, a description will be given of a technical effect obtained by the information processing system 10 according to the 17th example embodiment.

As described in FIG. 31 and FIG. 32, in the information processing system 10 according to the 17th example embodiment, when a skilled photographer who is accustomed to capture images captures the eye image, the quality criterion is raised at the time of registering the captured eye image as the registration image. In this way, it is possible to register the image of higher quality when asking a skilled photographer to capture images.

A processing method that is executed on a computer by recording, on a recording medium, a program for allowing the configuration in each of the example embodiments to be operated so as to realize the functions in each example embodiment, and by reading, as a code, the program recorded on the recording medium, is also included in the scope of each of the example embodiments. That is, a computer-readable recording medium is also included in the range of each of the example embodiments. Not only the recording medium on which the above-described program is recorded, but also the program itself is also included in each example embodiment.

The recording medium to use may be, for example, a floppy disk (registered trademark), a hard disk, an optical disk, a magneto-optical disk, a CD-ROM, a magnetic tape, a nonvolatile memory card, or a ROM. Furthermore, not only the program that is recorded on the recording medium and that executes processing alone, but also the program that operates on an OS and that executes processing in cooperation with the functions of expansion boards and another software, is also included in the scope of each of the example embodiments. In addition, the program itself may be stored in a server, and a part or all of the program may be downloaded from the server to a user terminal. The program may be provided to a user in a form of Saas (Software as a Service), for example.

### SUPPLEMENTARY NOTES

The example embodiments described above may be further described as, but not limited to, the following Supplementary Notes below.

### Supplementary Note 1

An information processing system according to Supplementary Note 1 is an information processing system including: an image capturing unit that captures an eye image including an iris of an image capturing target; and an output unit that outputs first guide information for capturing the eye image that allows iris recognition, to a photographer who is different from the image capturing target, in response to the eye image being captured.

### Supplementary Note 2

An information processing system according to Supplementary Note 2 is the information processing system according to Supplementary Note 1, further including a display unit that displays the first guide information, wherein the image capturing unit is disposed on a rear side of the display unit.

### Supplementary Note 3

An information processing system according to Supplementary Note 3 is the information processing system according to Supplementary Note 2, wherein the display unit is configured to display a captured image captured by the image capturing unit, and the first guide information includes information for displaying an iris capturing frame, which is superimposed on the captured image displayed on the display unit and in which the iris of the image capturing target is to be included.

### Supplementary Note 4

An information processing system according to Supplementary Note 4 is the information processing system according to Supplementary Note 3, wherein the iris capturing frame changes a state thereof from a non-displayed state to a displayed state, in a case of receiving an operation for a part around an eye of the image capturing target in the captured image displayed on the display unit, and the image capturing unit zooms the part around the eye such that the iris of the image capturing target has a size suitable for the iris recognition, in response to the iris capturing frame being in the displayed state.

### Supplementary Note 5

An information processing system according to Supplementary Note 5 is the information processing system according to Supplementary Note 3 or 4, wherein the first guide information includes information indicating a direction of moving an image capturing range of the image capturing unit, in order to include the iris of the image capturing target in the iris capturing frame.

### Supplementary Note 6

An information processing system according to Supplementary Note 6 is the information processing system according to Supplementary Note 1 or 2, further including a determination unit that determines whether or not the eye image satisfies an authenticable condition for allowing the iris recognition, wherein the output unit outputs the first guide information in response to a determination that the authenticable condition is not satisfied.

### Supplementary Note 7

An information processing system according to Supplementary Note 7 is the information processing system according to Supplementary Note 6, wherein the authenticable condition includes a plurality of conditions corresponding to a plurality of parameters, the determination unit determines whether or not the eye image satisfies the authenticable condition, for each of the plurality of parameters, and the output unit outputs the first guide information, based on a determination result for each of the plurality of parameters.

### Supplementary Note 8

An information processing system according to Supplementary Note 8 is the information processing system according to Supplementary Note 7, wherein the determination unit determines whether or not the eye image satisfies the authenticable condition, based on a threshold set for each of the plurality of parameters, and the output unit sequentially outputs the first guide information corresponding to each of the plurality of parameters, in order corresponding to a degree of deviation from the threshold.

### Supplementary Note 9

An information processing system according to Supplementary Note 9 is the information processing system according to Supplementary Note 7, wherein the output unit is configured to output second guide information for capturing the eye image that allows the iris recognition, to the image capturing target, in response to the eye image being captured, and the output unit selects and outputs at least one of the first guide information and the second guide information, based on a determination result for each of the plurality of parameters.

### Supplementary Note 10

An information processing system according to Supplementary Note 10 is the information processing system according to Supplementary Note 9, wherein the output unit outputs the first guide information and the second guide information in different aspects from each other.

### Supplementary Note 11

An information processing system according to Supplementary Note 11 is the information processing system according to Supplementary Note 9, wherein the output unit outputs the second guide information instructing the image capturing target to maintain a posture thereof, in a case of outputting the first guide information requiring adjustment, to the photographer.

### Supplementary Note 12

An information processing system according to Supplementary Note 12 is the information processing system according to Supplementary Note 9, wherein the output unit outputs the second guide information indicating that it takes time to start to capture the eye image, to the image capturing target, in a case of outputting the first guide information requiring a predetermined time or more of adjustment, to the photographer.

### Supplementary Note 13

An information processing system according to Supplementary Note 13 is the information processing system according to Supplementary Note 1 or 2, further including: an identification unit that identifies an image capturing cooperator located near the image capturing target; and a granting unit that grants a reward for capturing the eye image, to the image capturing cooperator authenticated by using the image capturing unit carried/owned by the image capturing target.

### Supplementary Note 14

An information processing system according to Supplementary Note 14 is the information processing system according to Supplementary Note 13, further including a determination unit that determines the photographer from among a plurality of image capturing cooperators, wherein the determination unit preferentially determines that the image capturing cooperator having experience of using the image capturing unit carried/owned by the image capturing target is the photographer.

### Supplementary Note 15

An information processing system according to Supplementary Note 15 is the information processing system according to Supplementary Note 13, wherein the output unit outputs the first guide information including information for making the image capturing unit carried/owned by the image capturing target, ready for image capturing, to the image capturing cooperator who captures the eye image.

### Supplementary Note 16

An information processing system according to Supplementary Note 16 is the information processing system according to Supplementary Note 13, wherein in a case where the image capturing cooperator who captures the eye image is a skilled photographer with a lot of experience of capturing the eye image, the output unit reduces and outputs an amount of information included in the first guide information, as compared to a case where the image capturing cooperator is not the skilled photographer.

### Supplementary Note 17

An information processing system according to Supplementary Note 17 is the information processing system according to Supplementary Note 13, further including a registration unit that registers the eye image as a registration image to be used for the iris recognition, in response to the eye image satisfying a predetermined quality criterion, wherein in a case where the image capturing cooperator who captures the eye image is a skilled photographer with a lot of experience of capturing the eye image, the registration unit raises the predetermined quality criterion a criterion, as compared to a case where the image capturing cooperator is not the skilled photographer.

### Supplementary Note 18

An information processing method according to Supplementary Note 18 is an information processing method that is executed by at least one computer, the information processing method including: capturing an eye image including an iris of an image capturing target; and outputting first guide information for capturing the eye image that allows iris recognition, to a photographer who is different from the image capturing target, in response to the eye image being captured.

### Supplementary Note 19

A recording medium according to Supplementary Note 19 is a recording medium on which a computer program that allows at least one computer to execute an information processing method is recorded, the information processing method including: capturing an eye image including an iris of an image capturing target; and outputting first guide information for capturing the eye image that allows iris recognition, to a photographer who is different from the image capturing target, in response to the eye image being captured.

### Supplementary Note 20

A computer program according to Supplementary Note 20 is a computer program that allows at least one computer to execute an information processing method, the information processing method including: capturing an eye image including an iris of an image capturing target; and outputting first guide information for capturing the eye image that allows iris recognition, to a photographer who is different from the image capturing target, in response to the eye image being captured.

The present disclosure is allowed to be changed, if desired, without departing from the essence or spirit of this disclosure which can be read from the claims and the entire specification. An information processing system, an information processing method, and a recording medium with such changes are also intended to be within the technical scope of the present disclosure.

### DESCRIPTION OF REFERENCE CODES

10 Information processing system
11 Processor
16 Output apparatus
18 Camera
50 Smartphone
60 Image capturing target
70 Image capturing cooperator
110 Image capturing unit
120 Guide information output unit
121 Selection unit
122 Adjustment determining unit
123 Skill level determining unit
130 Display unit
140 Determination unit
150 Identification unit
160 Reward granting unit
170 Photographer determining unit
180 Registration unit
181 Skill level determining unit
200 Iris
300 Pupil

What is claimed is:

1. An information processing system comprising:
at least one memory that is configured to store instructions; and
at least one processor that is configured to execute the instructions to:
receive an eye image for iris recognition by a camera; and
output first guide information in response to receiving the eye image to a photographer who is different from the target.

2. The information processing system according to claim 1, wherein the at least one processor is configured to execute the instructions to:
display the first guide information on a display; and
wherein a display direction of the display is different from a capturing direction of the camera.

3. The information processing system according to claim 2, wherein
the first guide information includes information that is superimposed on the image received by the camera and displayed on the display.

4. The information processing system according to claim 1, wherein
the first guide information prompts photographer to make an adjustment.

5. The information processing system according to claim 3, wherein
the first guide information includes information indicating a direction to move photographing position of the camera to include the iris in the frame.

6. The information processing system according to claim 1, wherein the at least one processor is configured to execute the instructions to:
determine whether or not the eye image satisfies an authenticable condition for the iris recognition; and
output the first guide information in response to a determining that the authenticable condition is not satisfied.

7. The information processing system according to claim 6, wherein
the authenticable condition includes a plurality of parameters, and
the at least one processor is configured to execute the instructions to:
determine whether or not the eye image satisfies each of the plurality of parameters; and
output the first guide information, based on determining that the eye image does not satisfy at least one of the plurality of parameters.

8. The information processing system according to claim 7, wherein the at least one processor is configured to execute the instructions to:
determine whether or not the eye image satisfies the authenticable condition, based on a threshold set for at least one of the plurality of parameters; and
output the first guide information corresponding to each of the plurality of parameters, in an order corresponding to a degree of deviation from the threshold of each of the plurality of parameters.

9. The information processing system according to claim 7, wherein the at least one processor is configured to execute the instructions to:
output second guide information to capture the eye image for the iris recognition, in response to receiving the eye image; and
select and output at least one of the first guide information and the second guide information, based on whether the eye image satisfies at least one of the plurality of parameters.

10. The information processing system according to claim 9, wherein
the first guide information and the second guide information have different characteristics.

11. The information processing system according to claim 9, wherein
the second guide information prompts the target to maintain a posture, after the first guide information prompts the photographer to make an adjustment.

12. The information processing system according to claim 9, wherein
the at least one processor is configured to execute the instructions to output the second guide information indicating that it takes time to start to capture the eye image, to the target, in response to outputting the first guide information requiring a predetermined time or more of adjustment, to the photographer.

13. The information processing system according to claim 1, wherein the at least one processor is configured to execute the instructions to:
identify the photographer who is located near the target based on mobile device of the target; and
grant a reward to the photographer after the photographer agrees to capture the eye image of the target.

14. The information processing system according to claim 13, wherein the at least one processor is configured to execute the instructions to:
select the photographer from a plurality of photographers based on an experience of using the camera.

15. (canceled)

16. The information processing system according to claim 13, wherein the at least one processor is configured to execute the instructions to,
determine an amount of information included in the first guide information based on an experience of photographer capturing the eye image.

17. The information processing system according to claim 13, wherein the at least one processor is configured to execute the instructions to:
register the eye image as a registration image to be used for the iris recognition, in response to the eye image satisfying a predetermined quality criterion; and
determine the predetermined quality criterion based on the experience of photographer capturing the eye image.

18. An information processing method that is executed by at least one computer, the information processing method comprising:
receiving an eye image for iris recognition by a camera; and
outputting first guide information in response to receiving the eye image, to a photographer who is different from the target.

19. A non-transitory recording medium on which a computer program that allows at least one computer to execute an information processing method is recorded, the information processing method including:
receiving an eye image for iris recognition by a camera; and
outputting first guide information in response to receiving the eye image, to a photographer who is different from the target.

20. The information processing system according to claim 3, wherein
the first guide information includes a frame within which an iris of the target should be located.

21. The information processing system according to claim 3, wherein the at least one processor is configured to execute the instructions to
change at least one of a photographing position or zoom of the camera to include the iris in the frame in response to receiving the action.
